(19) United States
(12) Patent Application Publication
(10) Pub. No.: US 2022/0202994 A1
(43) Pub. Date: Jun. 30, 2022

(54) BIOTIN-AVIDIN CONTROLLED DELIVERY SYSTEMS

(71) Applicant: Drexel University, Philadelphia, PA (US)

(72) Inventor: Kara Lorraine Spiller, Philadelphia, PA (US)

(21) Appl. No.: 17/570,681

(22) Filed: Jan. 7, 2022

Related U.S. Application Data
(63) Continuation of application No. 15/299,084, filed on Oct. 20, 2016, now abandoned.
(60) Provisional application No. 62/245,089, filed on Oct. 22, 2015.

(51) Int. Cl. A61L 27/54

(52) U.S. Cl. CPC A61L 27/54; A61L 2300/252; A61L 2300/426; C12N 2501/2304; C12N 2533/90; A61L 2430/06; A61L 2430/02; C12N 2537/10; C12N 2501/231

(57) ABSTRACT

The application relates to the delivery of immunomodulatory molecules, including cytokines, to the situs of tissue scaffolds, and wounds including injuries.

### CROSS-REFERENCE TO RELATED APPLICATIONS

This application claims the benefit of the priority of U.S. Provisional Patent Application No. 62/245,089, filed Oct. 22, 2015, which application is incorporated herein by reference in its entirety.

### STATEMENT REGARDING FEDERALLY SPONSORED RESEARCH OR DEVELOPMENT

This invention was made with government support under contract numbers: EB002520, DE016525 and AR061988 awarded by the National Institutes of Health. The government has certain rights in the invention.

### BACKGROUND OF THE INVENTION

A naturally occurring ligand-protein system that has been widely exploited in biotechnology is biotin-avidin or biotin-streptavidin. Avidin is a globular protein with four binding sites for the small molecule biotin, which binds with extremely high specificity and strength. The rate that biotin dissociates from avidin is so low (half life of 200 days) that it is considered essentially covalent. Streptavidin has similar structure to avidin, but binds more strongly to biotin when biotin is conjugated to another molecule.

Because biotin is small (244 g/mol), it can be conjugated to sensitive proteins and even cells without significantly damaging their bioactivity, in a process called biotinylation. Biotin-avidin interactions have been used in a diverse array of biotechnology applications, from separation chromatography to immunohistochemistry.

Wound healing remains a challenge in clinical medicine. M1 macrophages are often believed to be detrimental to healing, while M2 macrophages are believed to promote healing. This good-vs.-evil M1-M2 paradigm is believed by many in the biomaterials, regenerative medicine, and wound healing communities because chronic wounds with impaired healing also have persistently elevated levels of M1 macrophages. New systems for steering macrophage polarization in the wound healing process are needed.

Cartilage damage and cartilage-related diseases are the most common cause of disability in the United States today, occurring in approximately 20% of the population at a direct cost to the economy of $28.6 billion. Current treatments for arthritis include anti-inflammatory drugs for amelioration of symptoms and total joint replacement. Because cartilage lacks the capability for repair, tissue engineering strategies are essential.

Spinal cord injury and traumatic brain injury, as well as nervous system disorders, continue to pose a challenge in clinical medicine. New tools are needed to modulate the immune system to promote healing and to support medical interventions such as implantation of engineered nerve grafts, among other applications of the inventions described herein.

### SUMMARY OF THE INVENTION

By conjugating biotin to relatively bulky molecules like proteins, biotin's binding affinity for avidin or streptavidin, so that it dissociates much more quickly than it would in its free form. This aspect may be utilized in a number of applications wherein controlled release of a molecule from a composition is advantageous, including in tissue scaffold vascularization, including bone scaffold vascularization, vascularized tissue, wound treatment, and neuronal regeneration. By modulating the form of ‘avidin’, the form/derivative of biotin, and the length of the spacer arm separating biotin from the molecule to be conjugated and released, molecules of varying sizes can be delivered in a controlled manner. All of the above are considered as embodiments and are hereby incorporated within the Detailed Description, below.

### BRIEF DESCRIPTION OF THE DRAWINGS

FIG. 1A shows fluorescent streptavidin only binds to biotinylated scaffolds. FIG. 1B shows fluorescent streptavidin does not bind to non-biotinylated scaffolds. FIG. 1C shows biotinylated IL4 was released over 6 days in vitro (ELISA; n=4; representative data shown from an experiment repeated 3 times). Some error bars are too small to be seen. FIG. 1D shows biotinylated IL4 caused M2a polarization of seeded primary human macrophages in vitro, measured by gene expression of multiple M1 and M2a markers. The negative controls (left bars) in this experiment were scaffolds treated the same way but without IL4. All differences between negative control and biotin-IL4 were statistically significant (p<0.05, Student's t-test) [19]. FIG. 1E shows release of cytokines due to desorption. FIG. 1F shows biotinylated IL10 caused M2c polarization of seeded primary human macrophages in vitro, measured by expression of CD163 (*p<0.05).

FIG. 2A-2F show secreted proteins by polarized primary human macrophages M0, M1, M2a, and M2c, determined by enzyme-linked immunosorbent assays (ELISA) (n=4-6 donors, mean+/−SEM<*p<0.05, one way ANOVA with Tukey's post-hoc analysis). FIG. 2A shows VEGF secreted by polarized primary human macrophages. FIG. 2B shows PDGF-BB secreted by polarized primary human macrophages. FIG. 2C shows TIMP3 secreted by polarized primary human macrophages. FIG. 2D shows MMP7 secreted by polarized primary human macrophages. FIG. 2E shows MMP8 secreted by polarized primary human macrophages. FIG. 2F shows MMP9 secreted by polarized primary human macrophages.

FIG. 3A shows schematic representation showing that IL10 is displayed on the surface of PLGA nanoparticles using biotin-avidin interactions, which are strong but not covalent, causing the release of IL10 upon contact with the IL10 receptor IL l OR on the surface of macrophages. Macrophages, in close proximity due to their proclivity for phagocytosis, convert to the M2c phenotype and release anti-fibrotic factors like MMPs.

FIG. 3B shows Scanning electron micrograph (SEM) of sub-micron PLGA nanoparticles. Scale bar is 1 um.

FIG. 3C shows Primary human macrophages upregulate the M2c-specific marker CD163 when seeded on scaffolds conjugated with IL10 via biotin-avidin interactions.

FIG. 4A shows MMP (matrix metalloproteases) secretion by polarized macrophages in vitro.

FIG. 4B shows clustering analysis of gene expression after injury. Pie charts show relative contribution of phenotype markers to each cluster.

FIG. 5 shows a predicted release profile of biotinylated IL10 from porous scaffolds with D of 3.6×10⁵ um²/day, K_{D} of 10⁻⁶M (top curve) vs. K_{D} of 10⁻⁸M (bottom curve), and concentrations of IL10 and avidin of 4×10⁻⁵M and 1.33 ×10⁻⁵M, respectively.

FIG. 6 shows amount of fluorescent dextran in solution following addition of biotinylated dextran to scaffolds. ‘Control’ reflects scaffolds prepared with PBS instead of streptavadin prior to biotinylated dextran addition. ‘Biotin’ reflects scaffolds prepared with streptavadin prior to biotinylated dextran addition.

### DETAILED DESCRIPTION OF THE INVENTION

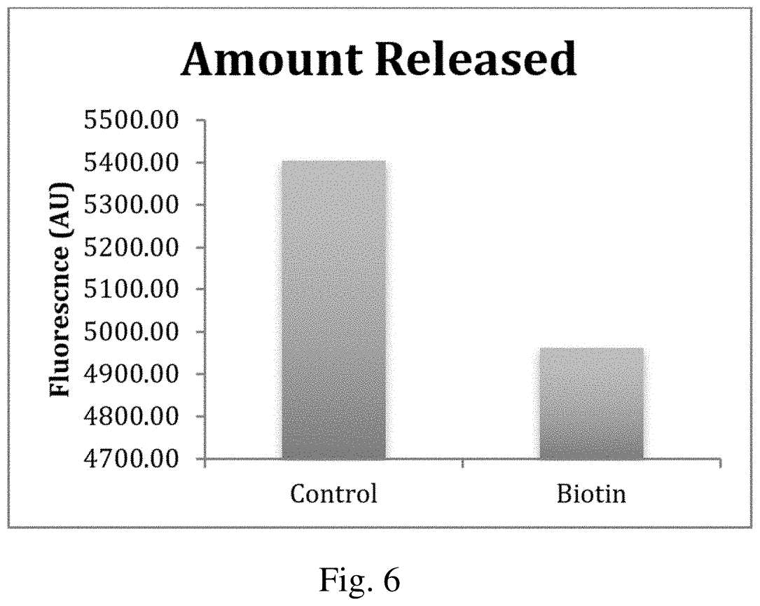

The release of a biotinylated molecule from a biotinylated surface (e.g., a cell, biomaterial, scaffold, or nanoparticle) may be defined by one or more formulae. When used herein, the terms “scaffold” and “biomaterial” may be used interchangeably, unless otherwise stated or apparent to one of skill in the art. Unless otherwise indicated, the term “biomaterial” in embodiments may include any synthetic or natural material suitable for use in constructing artificial organs or prostheses or to replace bone or tissue. Unless otherwise indicated, the term “scaffold” in embodiments may include any structure formed in whole or in part by a “biomaterial.” The release of biotinylated proteins from biomaterials depends on affinity interactions and diffusion. In one embodiment, the following Equation 1 is a useful mathematical model useful in the design of formulations/compounds that result in release of the biotinylated molecule of interest from the biotinylated surface.

$$\frac{\partial [ B ]}{\partial t} = \frac{D}{r} \frac{\partial }{\partial r} ( r \frac{\partial [ B ]}{\partial r} ) + D \frac{{\partial }^{2} [ B ]}{\partial {z}^{2}} - {k}_{on} [ A ] [ B ] + {k}_{off} ( [ AB ] ) \qquad Equation 1$$

wherein r is the radius and z is the height of a cylindrical biomaterial, [B] is the concentration of biotinylated molecule (e.g., biotinylated IL10), and [A] is the concentration of avidin-like protein (e.g., avidin) at any time t. [AB] is the concentration of the avidin-like protein-biotin complex at time t, which can be determined by subtracting the concentration of released avidin-like protein [A] from the starting concentration [A]ₒ and assuming that any remaining avidin-like protein in the system is bound to biotin. The biotinylated IL10-avidin-like-protein-biotinylated scaffold interaction is assumed to be a single bond to simplify the model. D is the diffusion coefficient of biotinylated molecule in the scaffold or tissue, which can be determined experimentally by using non-biotinylated scaffolds and/or biotinylated scaffolds without avidin. The on and off rates (kₒₙ and k_{off}) are derived from SPR experiments or by approximation through fitting Equation 1 to release curves of biotinylated IL10. The completed Equation 1 may be used to describe the extent of control over protein release for a range of preparation parameters. See FIG. 6.

The on and off rates (kₒₙ and k_{off}) are determined experimentally using surface plasmon resonance (SPR). Changes in the SPR signal upon adsorption to a layer of immobilized avidin-like protein can be directly measured.

In one or more further embodiments, additional constants or factors may be introduced into Equation 1 to account for other variables such as scaffold dissolution, the presence or absence of free biotin, and/or other variables known to those of skill in the art or identified through the teachings herein. In further embodiments, these constants or factors account for factors of impact in in vivo versus in vitro application, and in humans, mammals, companion animals, or production/food animals, versus in vitro or animal models.

Affinity-based drug delivery systems have been employed in tissue engineering to control the release of proteins. For example, heparin binds to various growth factors, allowing sustained release of these growth factors from scaffolds that contain heparin. However, these systems are limited to only a handful of affinity binding pairs. Because avidin has four binding sites for biotin, any molecule can be biotinylated and joined to another molecule via an avidin bridge. Moreover, because there are so many commercially available biotinylation reagents, even researchers without experience in bioconjugation techniques can employ this technology.

As used herein, the term “avidin-like protein” refers to avidin, derivatives thereof, streptavidin, derivatives thereof, whether naturally occurring or synthetic, recombinant, or artificial. Unless otherwise indicated by statement or by context, the terms “avidin-like protein” and “avidin” are interchangeable. Similarly, unless otherwise indicated, embodiments referring to streptavidin or derivatives thereof, also define embodiments where avidin or avidin derivatives are utilized, and vice versa. These embodiments include nitroavidin (CAPTAVIDIN™ protein reagent, Thermo Fisher Scientific), a form of avidin in which the tyrosine residues near the biotin-binding site have been nitrated, increasing the dissociation constant (K_{D}) of biotin from 10⁻¹⁵M to 10⁻⁶ M. Still further, other streptavidin mutants such as those previously described [including but not limited to those described in [7, 9] may be utilized. As used herein the term “biotin” refers to biotin, derivatives thereof, and whether naturally occurring or synthetic, recombinant, or artificial. Any embodiment described herein with respect to any biotin describes an embodiment utilizing any other biotin described herein or known to one of skill in the art. Biotin derivatives include iminobiotin and desthiobiotin. Desthiobiotin is a sulfur-free, single-ring analog of biotin that binds avidin with equal specificity but substantially decreased affinity, increasing the dissociation constant from 10⁻¹⁵ M to 10⁻¹¹ M in its free form, and even more when conjugated to larger molecules.

The spacer arm (or spacer) connecting biotin to the molecule of interest may in some embodiments be absent. However, in many embodiments herein, the spacer arm will be present and have a length of between 1 and 100 angstroms, between 10 and 60 angstroms, between 13.5 angstroms and 56 angstroms, or any range, integer, or fraction within these ranges. For example, the following spacers may be utilized: Sulfo-NHS (13.5 Å), Sulfo-NHS-LC (22.4 Å), Sulfo-NHS-LC-LC (30.5 Å), NHS-PEG4 (29 Å), and NHS-PEG12 (56 Å). PEG4 and PEG12 refer to polyethylene glycol (PEG) and the number of same. PEG4 refers to a four-unit PEG group. In other embodiments, PEG, PEG2, PEGS, PEGS, PEG6, PEG7, PEG8, PEGS, PEG10, PEG11, PEG15, PEG20, PEG30, PEG40, PEG50, or any range of PEGs within the span of one or more of those integers, inclusive. However, the invention is not so limited. Sulfo-NHS is N-hydroxysulfosuccinimide. LC refers to ‘long chain’, which may be a 6-atom chain extension of the valeric acid group of biotin. Solfo-NHS-LC-Biotin is succinimidyl-6-(biotinamido)hexanoate.

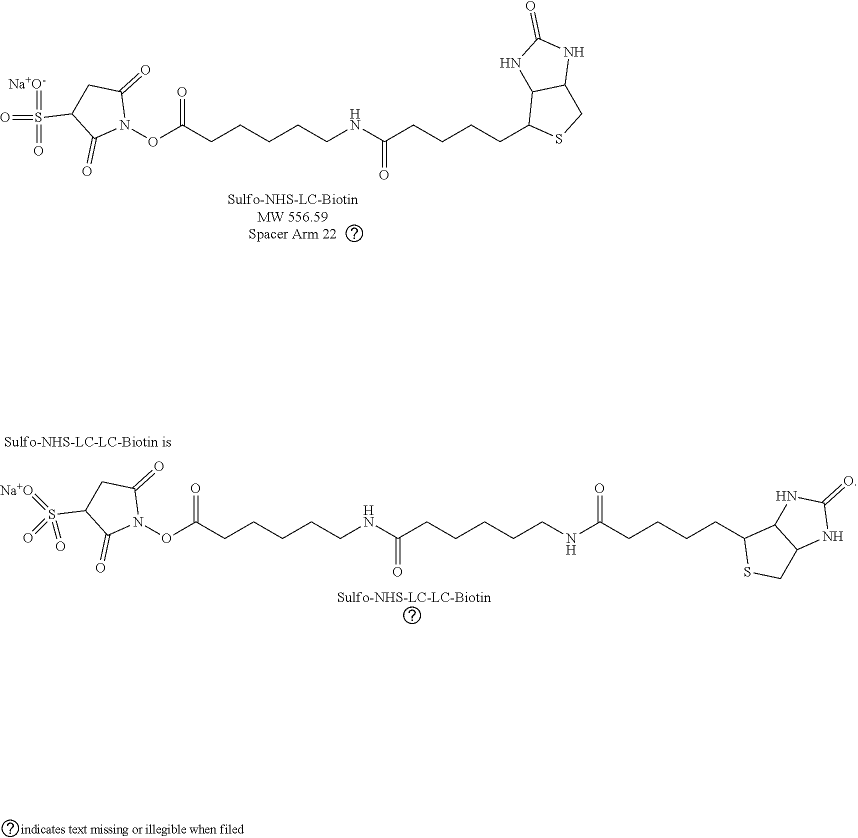

A non-limiting list of examples of variables which may be combined in generating one or more appropriate release profiles is found in Tables 1 and 2 as follows.

TABLE 1
Biotinylation variables

| Property | Variable | Outcome |
|---|---|---|
| Avidin | Avidin vs. | Increasing |
| form | streptavidin vs. | dissociation of |
| | CaptAvidin | biotinylated protein |
| | | from CaptAvidin |
| | | followed by avidin |
| | | and streptavidin |
| Biotin | Biotin vs. | Increased |
| derivatives | desthiobiotin | dissociation with |
| | | desthiobiotin. |
| Length of | Sulfo-NHS (13.5 | Decreased |
| spacer arm | Å), Sulfo-NHC-LC | dissociation with |
| | (22.4 Å), Sulfo- | increasing |
| | NHS-LC-LC (30.5 | molecular weight |
| | Å), NHS-PEG4 (29 | of spacer arm. |
| | A Å, NHS-PEG12 | |
| | (56 Å) | |
| Size of | Biotinylated | Increased |
| conjugated | fluorescein (0.3 | dissociation with |
| molecule | kDa) vs. IL4 (17 | increased |
| | kDa) | molecular weight |
| | | of bound molecule. |

TABLE 2
Experimental biotinylation variables to be tested
in Objective 1 and expected outcomes.

| | | Experimental | |
|---|---|---|---|
| | Property | Variable | Outcome |
| Main | Size of | Fluorescent | Increased dissociation with |
| variables | conjugated | dextran of | increased molecular weight |
| | molecule | molecular | of bound molecule. |
| | | weight 3, 10, | |
| | | 70, 500, and | |
| | | 2000 kDa | |
| | Length of | 13.5, 22.4, 29, | Decreased dissociation |
| | spacer arm | 30.5, 56 Å | with increasing molecular |
| | | (linking biotin | weight of spacer arm. |
| | | to NHS) | |
| Secondary | Biotin | Biotin vs. | Increased dissociation with |
| variables | derivatives | desthiobiotin | desthiobiotin. |
| | Avidin | Avidin vs. | Increasing dissociation |
| | form | streptavidin vs. | from nitroavidin followed |
| | | nitroavidin | by avidin and streptavidin |

As used herein the terms, “molecule”, “molecule of interest”, or “conjugated molecule” refers to any cargo that may be linked to biotin (directly or via a spacer). Any number of such molecules may be bound in compositions of the invention and delivered/released according to methods of the invention. The molecule is not limited to the embodiments described herein, but may be extended to include any molecule or other cargo which one of skill in the art would deliver based on the compositions, methods, and strategies described herein.

The molecule/cargo may have a molecular weight between 1 and 5000 kDa, between 1 and 2000 kDa, between 3 and 500 kDa, between 10 and 500 kDa, or any range, integer, or fraction, within these ranges.

The molecule/cargo may be interleukin 4 (IL4). The molecule may be IL10. In other embodiments, the molecule may be VEGF, PDGFB, PDGF-BB, PDGFA, PDGF-AB, IL3, IL-6, IL13, MCP1 (aka CCL2), TGF-β, an immune complex, lipopolysaccharide (LPS), a glucocorticoid, interferon gamma (IFNg or IFNγ), TGF-beta, leukocyte inhibitory factor, or macrophage chemotactic factor. In still further embodiments, anti-inflammatory drugs including dexamethasone may be used. Other anti-inflammatory drugs include steroids generally, such as prednisone and hydrocortisone. Other molecules include non-steroidal anti-inflammatory drugs (NSAIDS), including aspirin, ibuprofen, and naproxen, and immune selective anti-inflammatory derivatives (ImSAIDS). Still other molecules referenced in this application or known to those of skill in the art, including for a purpose described herein, are contemplated.

In one embodiment, a compound comprises a molecule or cargo bound to a biotin. In a further embodiment, the molecule or cargo is bound to a biotin through a spacer arm or linker as described herein.

In another embodiment, a compound comprises a molecule or cargo bound to a biotin, which biotin is bound to an avidin. In a further embodiment, the molecule or cargo is bound to a biotin through a spacer arm or linker as described herein.

It is also contemplated in embodiments of this application that two or more molecules/cargoes are administered as part of one or more compounds of the invention, e.g., according to Equation 1. For example, where one molecule (e.g., IL4) is desired to be released several hours or days or other interval after a first molecule (e.g., IFN-gamma), two compounds may be prepared for linkage to a biotinylated surface (e.g., a cell, scaffold, or nanoparticle. In that way, sequencing may be permitted such that the cargoes will be delivered optimally to suit the subject's immune system or regenerative capabilities. One or more compounds may be delivered in a pharmaceutical composition as described herein.

In one embodiment, nanoparticles include any microscopic particle having at least one dimension less than 100 nm. Nanoparticles used for medical applications will be biocompatible (able to integrate with a subject without eliciting immune response or any negative effects) and will be nontoxic or of appropriate limited toxicity (substantially harmless to a given subject). Selection of an appropriate nanoparticles may be made by one of skill in the art based upon hydrodynamic size, shape, amount, surface chemistry, the route of administration, reaction of the immune system (especially a route of the uptake by macrophages and granulocytes) and residence time in the bloodstream of the subject.

Any nanoparticle or nanomaterial referenced in the literature for administration to a subject, including in association with immune response, tissue growth, or nervous system regeneration may be used. Nanoparticles may include liposomes, including solid lipid nanoparticles, nanostructured lipid carriers, or lipid drug conjugates. Polymeric nanoparticles are also contemplated, including those obtained from synthetic polymers such as poly-e-caprolactone, polyacrylamide and polyacrylate, or natural polymers, e.g., albumin, a nucleic acid including DNA or RNA, chitosan, or gelatin. Dendrimer nanocarriers may also be used, including poly(amido amide). Still further silica materials may be used including xerogels, mesoporous silica nanoparticles, including MCM-41, SBA-15. In still further embodiments, carbon nanomaterials are contemplated. Still further, magnetic nanoparticles may be used, including iron oxide nanoparticles.

Any implanted device, including engineered tissues, will inevitably interact with the inflammatory response, especially when implanted into a diseased environment. In arthritis, macrophage-derived inflammatory cytokines promote breakdown of cartilage tissue in vivo and in vitro, while promoting osteogenic differentiation of mesenchymal stem cells (MSCs). The induction of anti-inflammatory cytokines IL4 and IL10, which directly affect macrophage behavior, ameliorated symptoms of arthritis in multiple animal models. The corners of engineered cartilage constructs became mineralized when implanted subcutaneously in nude mice in vivo. Similarly, stress concentrations at areas of flexion in bioprosthetic heart valves cause structural damage that stimulates inflammation and ultimately leads to valve calcification and failure. Surprisingly, the mechanism of cardiac valve calcification is endochondral bone formation, with mesenchymal cells derived from multiple cell types differentiating into chondrocytes and then osteoblasts that participate in active bone remodeling, concomitant with blood vessel invasion. Inflammation can damage engineered tissues and lead to ectopic ossification of cartilage.

In many applications, the induction of macrophages enhances tissue regeneration because of the diverse growth factors that they secrete. The balance of beneficial and adverse effects of inflammation on engineered tissues appears to be related to the activation state of macrophages, which can change their phenotype rapidly from pro-inflammatory to anti-inflammatory based on environmental conditions.

In response to injury, macrophages rapidly switch their behavior from predominantly pro-inflammatory (often called M1) in the early stages of healing (0-3 days) to a state that promotes resolution of inflammation and healing (often called M2) at later stages (4-18 days). M1 macrophages initiate angiogenesis, a critical part of wound healing, and scaffolds that rapidly released M1-promoting cytokines are more vascularized in vivo than control scaffolds. M1 macrophages also stimulate bone regeneration, through their effects on angiogenesis and also direct stimulation of MSC osteogenesis. Until recently, it was believed that M1 macrophages were detrimental for tissue regeneration because chronic wounds contain higher levels of M1 macrophages than acute wounds. However, M1 and M2a macrophages work synergistically and sequentially to stimulate and stabilize nascent blood vessels. Importantly, the timing of activation of these phenotypes in normal wound healing is tightly regulated, with aberrations resulting in pathology. For example, my group and others have shown that excessive M1 activation is associated with impaired wound healing in human diabetic ulcers, while hybrid M1/M2a activation leads to scarring and fibrous encapsulation of biomaterials.

M2 macrophages can be further subdivided into two different phenotypes, M2a and M2c, which are induced in vitro by IL4 and IL10, respectively. While most studies fail to distinguish between these subtypes, M2a and M2c macrophages behave very differently in regulating tissue regeneration, with distinct roles that contribute to the process in different ways. M2a macrophages secrete high levels of cytokines associated with tissue deposition, especially platelet-derived growth factor-BB (PDGF-BB), while M2c macrophages secrete high levels of cytokines associated with tissue remodeling, including matrix metalloprotease-9 (MMP9). PDGF-BB also stimulates cartilage growth, while MMP9 stimulates cartilage breakdown. M2a macrophages inhibit while M2c macrophages promote endothelial cell sprouting in vitro.

Controlled manipulation of macrophage phenotype can be used to promote specific host responses in vivo, including angiogenesis and osteogenesis (M1), extracellular matrix deposition (M2a), and matrix remodeling (M2c). In one embodiment, tissue engineering strategies that inhibit M1 and M2c activation while promoting M2a activation enhance the growth and survival of engineered cartilage tissue.

Sequential M1 and M2 activation of macrophages is required for diabetic ulcer healing in human patients. This strategy is also applicable to other pathological situations in which sustained M1 macrophage activity has been described, including chronic venous ulcers, atherosclerotic lesions, traumatic spinal cord injury, and inflammatory renal disease. The platform technology described here could be used to release any protein or drug from a biomaterial or engineered tissue, and therefore would be useful for inducing cell infiltration into scaffolds from surrounding tissue, for example to promote blood vessel in growth, bone regeneration, or innervation. In addition, the system is reloadable as long as biotin remains bound to the biomaterial, so that a later infusion of avidin and biotinylated drug or protein would attach to the biotinylated material for subsequent release dictated by the affinity binding interactions.

In one embodiment, a method of treating a wound of a subject is provided comprising the sequential induction of M1 macrophages and M2 macrophages. In a further embodiment, the M1 macrophages induced are M1a, M1b, or M1a and M1b macrophages. The induced M2 macrophages induced are one or more of M2a, M2b, M2c, and M2d macrophages. In a further embodiment, the M2 macrophages induced are one or more of M2a, M2b, and M2c. In still a further embodiment the macrophages induced are M2a and M2c. In another embodiment, M2 macrophages are induced without induction of, or before induction of, M1 macrophages. In further embodiments, M2c macrophages are induced.

By sequential induction, the M2 macrophages may be induced in a subject from 1 to 12 months, 1 to 2 months, 1 to 4 weeks, 1 to 2 weeks, 2 to 14 days, 2 to 7 days, or 3 to 4 days, inclusive, following induction of M1 macrophages. In further embodiments, the M2 macrophages may be induced in a subject 2, 3, 4, 5, 6, 7, 8, 9, 10, 11, 12, 13, or 14 days, or 1 week, 2 weeks, 3 weeks, 4 weeks, 1 month, 2 months, 3 months, 4 months, 5 months, 6 months, 7 months, 8 months, 9 months, 10 months, 11 months, or 12 months following induction of M1 macrophages. In still further embodiments, the M2 macrophages may be induced to a subject in any combination of months, days, hours, minutes, and seconds within these ranges. For example, the M2 macrophages may be induced to a subject 2 days, 6 hours, following induction of the M2 macrophages.

In further embodiments, the induction of M1 macrophages is sequenced or repeated prior to induction of M2 macrophages. For example, M1b macrophages may be induced 1, 2, 3, 4, 5, 6, 7, 8, 9, 10, 11, 12, 13, or 14 days following induction of M1a macrophages. In still further embodiments, the Mlb macrophages may be induced in a subject in any combination of hours, minutes, and seconds from 1 to 13 days, or any range or sub-range thereof, following induction of M1a macrophages. Instead or, or in addition to, any of the above sequencing, induction of M1a and/or M1b may be repeated 1, 2, 3, 4, 5, or more times, from 1, 2, 3, 4, 5, 6, 7, 8, 9, 10, 11, 12, 13, or 14 days from first induction, and spaced at any combination of days, hours, minutes, and seconds from 1 to 13 days, or any range or sub-range thereof.

Likewise, the induction of M2 macrophages is sequenced or repeated. For example, M2b macrophages may be induced 1, 2, 3, 4, 5, 6, 7, 8, 9, 10, 11, 12, 13, or 14 days following induction of M2a macrophages. M2c macrophages may be induced 1, 2, 3, 4, 5, 6, 7, 8, 9, 10, 11, 12, 13, or 14 days, or 1 week, 2 weeks, 3 weeks, 4 weeks, 1 month, 2 months, 3 months, 4 months, 5 months, 6 months, 7 months, 8 months, 9 months, 10 months, 11 months, or 12 months following induction of M2a macrophages, or following induction of M2b macrophages. M2a macrophages may also be induced following induction of M2c macrophages, or of M2b macrophages, in any of the sequencing or repeating described above. In still further embodiments, the M2b and/or M2c macrophages may be induced in a subject in any combination of months, weeks, days, hours, minutes, and seconds from 1 to 12 months, 1 to 4 weeks, or 1 to 13 days, or any range or sub-range within these periods, following induction of other M2 macrophages. Instead or, or in addition to, any of the above sequencing, induction of M2a, M2b and/or M2c may be repeated 1, 2, 3, 4, 5, or more times, from 1, 2, 3, 4, 5, 6, 7, 8, 9, 10, 11, 12, 13, or 14 days, or 1 week, 2 weeks, 3 weeks, 4 weeks, 1 month, 2 months, 3 months, 4 months, 5 months, 6 months, 7 months, 8 months, 9 months, 10 months, 11 months, or 12 months, from first induction of an M2 macrophage, and spaced at any combination of months, weeks, days, hours, minutes, and seconds from 1 to 12 months, 1 to 4 weeks, or 1 to 13 days, or any range or sub-range thereof.

Still further provided are uses of compounds described herein in the treatment of a wound. Still further provided are uses of the compounds in the preparation of medicaments useful in the treatment of a wound. Still further provided is the use of one of more of the compounds described herein to treat a wound. The treatment may comprise any of the methods described herein.

The methods described herein may be used for any type of wound. In specific embodiments, the wound may be a chronic wound, an acute wound, an open wound, a closed wound, a clean wound, a contaminated wound, an infected wound, a diabetic wound, an ulcer, a diabetic ulcer, a foot sore, or a skin sore. However, the embodiments described herein are not so limited.

The wound may be acute. The wound may be chronic, hard-to-heal, or refractory. The wound may also be associated with a metabolic disease or other disease in which natural healing is diminished. The wound may be a diabetic wound. The wound may be a diabetic ulcer. The wound may be a wound of a patient with Type I or Type II diabetes.

The subject or patient may be a mammal, or more specifically a human. In further embodiments, still any subject or patient as defined or otherwise described herein may be treated according to the invention. Any composition described herein may be formulated for one or more species of subject or groups of subjects.

Monocytes may be polarized into M1, including M1a, M1b, and M2, including M2a, M2b, M2c, and M2d, macrophages using polarizing factors known to one of skill in the art. For polarization to Ml, interferon gamma and/or lipopolysaccharide (LPS) and/or TNF (tumor necrosis factor)-alpha may be used. In a further embodiment, interferon gamma is used. In still a further embodiment, interferon gamma and LPS are used. For polarization to M2a, IL(interleukin)-4 or IL-3 may be used, either separately or in combination. For polarization to M2b, immune complexes, LPS, or glucocorticoids may be used, either separately or in any combination. For M2c, IL-10, TGF-beta, or glutocorticoids may be used, either separately or in any combination. For M2d, IL-6, leukocyte inhibitory factor, macrophage chemotactic factor, or VEGF. Still other factors known in the art may be used to polarize macrophages, and the factors used are not intended to limit this application.

These compounds may be administered alone, as pharmaceutical compositions in combination with diluents and/or carriers and/or buffers and/or other components, including other compounds described herein. In one embodiment, they may be administered in saline. In another embodiment, in a hydrogel. Among other formulations, both saline and hydrogel formulations are contemplated for delivery by injection. Other components may include cytokines, cells, or other agents conventionally used to promote wound treatment or healing. Compositions may include stabilizers, antioxidants, and/or preservatives. Compositions may include, e.g., neutral buffered saline or phosphate buffered saline.

Carriers may include pharmaceutically acceptable material, composition or carrier, such as a liquid or solid filler, stabilizer, dispersing agent, suspending agent, diluent, excipient, thickening agent, solvent or encapsulating material, involved in carrying or transporting a compound or molecule useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. Carriers also includes any and all coatings, antibacterial and antifungal agents, and absorption delaying agents, and the like that are compatible with the activity of the components, e.g., cells, to be delivered, and are physiologically acceptable to the patient. Supplementary active compounds may also be incorporated into the compositions. Pharmaceutically acceptable salt of the compound or molecule useful within the invention.

Other ingredients that may be included are excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Still other additional ingredients that may be included in the pharmaceutical compositions used in the practice of the invention and are known in the art and described elsewhere.

Effective amounts of the compound(s) and other aspects of a pharmaceutical composition may be determined by one of skill in the art, including by a physician. Such amounts may be determined by with consideration of the age and/or weight of a patient, and further by the size, condition, location, and/or severity of the wound or wounds to be treated, including those as described in [79] International Patent Application No. WO 2015/077401.

Still further provided are kits comprising one of more of the components described herein in one or more vials, tubes, or other suitable vessels. The kit may further comprise a syringe or other medical instrument suitable to deliver a composition to a subject.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

Each term used herein will include embodiments where the term has been defined expressly or by usage in the literature, including within any of the documents incorporated by reference herein.

As used herein, the singular form of a term is intended to encompass the plural, and vice versa, unless otherwise noted.

All ranges referred to herein include all sub-ranges, integers, and fractions of integers, unless otherwise provided.

The terms “comprising,” “comprises,” “contains,” “containing,” “has,” “have,” “having,” “include,” includes,” “including”, and the like, are used interchangeably and indicate that the subject is open ended, unless otherwise noted.

The terms “consist,” “consists,” “consisting,” and the like, are used interchangeably and indicate that the subject is open ended, unless otherwise noted.

Throughout this application, where compositions, components, methods, or steps are described as required in one or more embodiments, additional embodiments are contemplated and are disclosed hereby for fewer compositions, components, methods, or steps, and for fewer compositions, components, methods, or steps in addition to other compositions, components, methods, or steps. All compositions, components, methods, or steps provided herein may be combined with one or more of any of the other compositions, components, methods, or steps provided herein unless otherwise indicated.

The terms “subject” and “patient” are, unless otherwise noted, used interchangeably to refer to the target or recipient of a treatment or composition described herein. These terms include, unless otherwise specified, all vertebrates, including all mammals, including humans. Unless otherwise noted, an embodiment using the term “subject” and “patient” is intended to include an embodiment directed solely to solely to mammals, solely to humans, solely to non-human mammals, solely to companion mammals, solely to companion vertebrates, solely to companion mammals, solely to non-human animals, and solely to non-human mammals. Unless otherwise indicated, reference to any of the above terms includes embodiments directed to the others.

The term “wound” is intended to refer to an injury to living tissue of a subject. Unless otherwise noted, embodiments referring to a wound include embodiments where the wound is one in which the skin is cut or broken, including surgical wounds. Further, embodiments referring to a wound include embodiments where the epidermis is broken. Still further, embodiments referring to a wound include embodiments where the dermis is cut or broken. In still other embodiments referring to a wound, the wound is a tissue other than of the skin, including internal surgical wounds. Unless otherwise noted, embodiments referring to a wound include embodiments where the wound is an ulcer.

The term “acute wound” refers to a wound which heals consistent with the timing or process conventional to the type and severity of the wound for the species of the subject.

The term “chronic wound” refers to a wound which does not heal consistent with the timing or process conventional to the type and severity of the wound for the species of the subject.

The terms “hard-to-heal” or “refractory” refer to a wound which does not heal using conventional therapies available as of the filing date of this application.

The term “diabetic wound” refers to any wound in an individual having diabetes. The terms “disease”, “disorder”, or “condition” are used herein to refer to any manifestations, symptoms, or combination of manifestations or symptoms, recognized or diagnosed as connected with a chronic wound, hard-to-heal wound, or diabetic wound.

The terms “treat,” “treating,” “treatment,” and the like, as used herein, refer to any method or composition used to reduce, improve, alleviate, ameliorate, or reduce the severity of, a wound or condition as defined herein.

The term “wound composition” refers to a composition that may be applied to a wound to promote healing or prevent further injury.

The term “pharmaceutically acceptable carrier” or “diluent” is intended to include any and all solvents, dispersion media, coatings, antibacterial and antifungal agents, isotonic and absorption delaying agents, adjuvants and the like, compatible with administration to humans. In one embodiment, the diluent is saline or buffered saline.

The term “effective amount,” unless otherwise noted, means an amount which provides a therapeutic benefit to a subject.

Specific embodiments of the invention include methods of modulating the immune response to a tissue scaffold, comprising biotinylating IL4 or IL10, biotinylating a scaffold, and linking the biotinylated IL4 or IL 10 to the scaffold via avidin or streptavidin. The IL4 or IL10 may be biotinylated via a spacer arm. A spacer arm may be sulfo-NHS, Sulfo-NHS-LC, Sulf-NHS-LC-LC, NHS-PEG4, or NHS-PEG12. Biotinylation may be with biotin, desthiobiotin or iminobiotin. A tissue scaffold may be for cartilage. A tissue scaffold may be for bone. Further provided are methods of delivering IL4 or IL10 to a macrophage, comprising biotinylating IL4 or IL10, biotinylating a nanoparticle, linking the biotinylated IL4 or IL10 to the nanoparticle, and delivering the nanoparticle to the macrophage. The IL4 or IL10 may be biotinylated via a spacer arm. A spacer arm may be sulfo-NHS, Sulfo-NHS-LC, Sulf-NHS-LC-LC, NHS-PEG4, or NHS-PEG12. Biotinylation may be with biotin, desthiobiotin or iminobiotin. A tissue scaffold may be for cartilage. A tissue scaffold may be for bone.

Still further provided are methods of modulating the immune response to a tissue scaffold, comprising biotinylating interferon gamma, biotinylating a scaffold, and linking the biotinylated interferon gamma via avidin or streptavidin. The methods may further comprise biotinylating IL4 or IL10 and linking the biotinylated IL4 or IL10 to the scaffold via avidin or streptavidin.

The embodiments described above further include that matter contained within the following examples, the claims, and any other component of the application.

### EXAMPLES

The invention is now described with reference to the following examples. These examples are provided for the purpose of illustration only and the invention should in no way be construed as being limited to these examples but rather should be construed to encompass any and all variations that become evident as a result of the teaching provided herein. The specific embodiments described in the Examples are intended to be embodiments of the invention.

### Example 1

### Release of Biotinylated Protein from Biotinylated Scaffolds

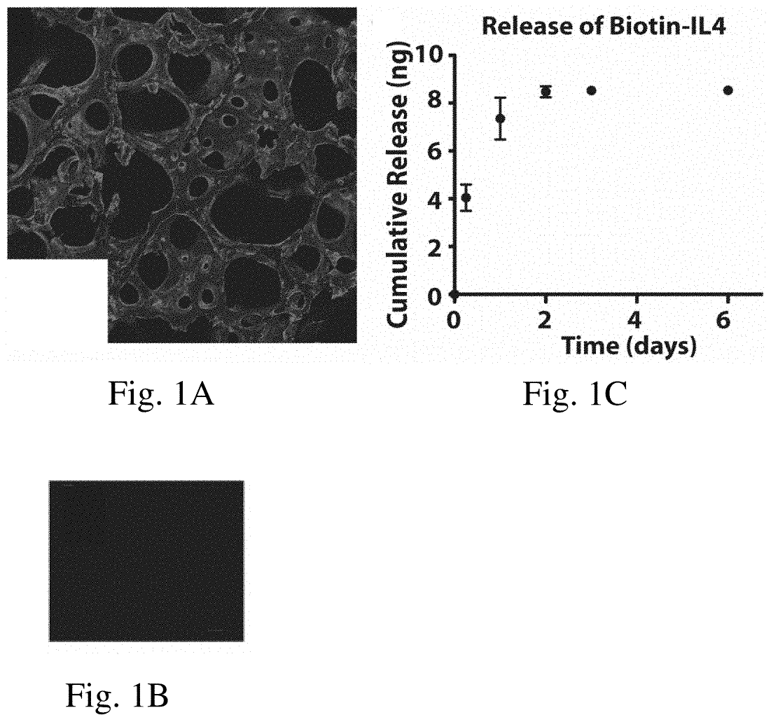

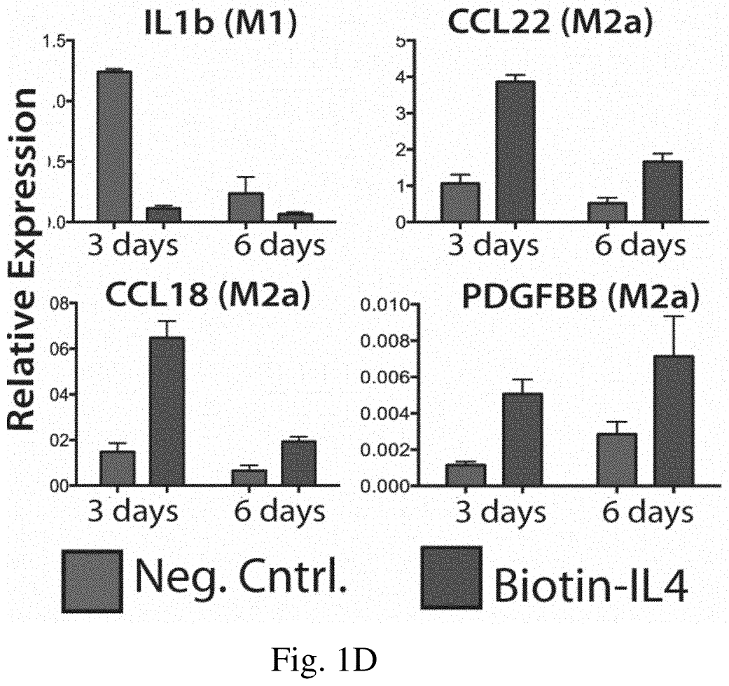

Decellularized bone scaffolds, prepared by washing cores obtained from the femurs of young cows with detergents and DNA-degrading enzymes, were biotinylated using the EZ-LINK® protein reagents, i.e., a family of biotin attached to protein-reactive moieties, such as NHS (N-hydroxysulfosuccinimide) in this case [19]. Even after multiple washes, fluorescently labeled streptavidin bound strongly to biotinylated bone scaffolds (FIG. 1a), but not to non-biotinylated scaffolds (FIG. 1b). The cytokine IL4, which promotes the M2a phenotype of macrophages, was biotinylated using similar techniques and joined to the scaffold with streptavidin. IL4 was gradually released over 6 days in vitro (FIG. 1c), while non-biotinylated cytokines were released rapidly, within 6 hours (FIG. 2e). This rate of protein release is much slower than that due to diffusion, but much faster than release that would be expected to result from biotin-streptavidin interactions in the absence of free biotin. Most likely, surface degradation of the scaffold combined with dissociation of biotin-avidin contributed to the release of biotinylated IL4. Human macrophages seeded on these scaffolds strongly polarized to the M2a phenotype (FIG. 1d), indicating the potential for this strategy to modulate macrophage behavior. Human macrophages seeded on scaffolds with biotinylated IL10 polarized to the M2c phenotype, as indicated by gene expression of CD163 (FIG. 1f), indicating the potential for this strategy to modulate macrophage behavior.

Macrophages release multiple growth factors depending on their phenotype. Therapeutic strategies aimed at controlling macrophage behavior can have major downstream effects on tissue healing. Understanding the effects of macrophage phenotype on the growth and survival of engineered tissue is the key to harnessing the inflammatory response for therapeutic strategies.

### Example 2

### Distinct Roles of Different Macrophage Phenotypes in Tissue Regeneration

Next generation sequencing of the transcriptomes of macrophages polarized in vitro to the M1, M2a and M2c phenotypes plus an unactivated control (M0) was performed. M1 macrophages expressed many genes encoding pro-inflammatory cytokines as well as the potent pro-angiogenic factor vascular endothelial growth factor (VEGF), as expected. Interestingly, M2a macrophages expressed genes associated with tissue deposition, including PDGF-BB and tissue inhibitor of metalloprotease-3 (TIMP3) (confirmed on the protein level in FIGS. 2B and 2C). PDGF-BB is a potent inducer of cartilage growth in vitro and in vivo, and TIMP3-deficient exhibit increased cartilage breakdown and hypertrophy. M2c macrophages upregulated multiple genes associated with matrix degradation, especially MMP7, MMP8, and MMP9. While matrix remodeling is an important part of normal tissue homeostasis and healing, excessive levels of MMPs are highly associated with cartilage degeneration in arthritis. M1 and M2c macrophages contribute to cartilage tissue breakdown, while M2a macrophages would support cartilage growth.

### Example 3

### Identification of Biotin Conjugation Parameters Impact on Biotin's Dissociation from Avidin.

Protein biotinylation—Proteins are biotinylated (or desthiobiotinylated) using the EZ LINK® protein reagents available from Thermo Fisher Scientific by incubating with a 20- to 50-fold molar excess of biotin-NHS for 2hrs according to the manufacturer's instructions. Biotinylated proteins will be dialyzed against PBS to remove unreacted biotin. The degree of biotinylation can be measured as previously described [19], by adding samples to a solution of avidin bound to the HABA dye (4′-hydroxyazobenzene-2-carboxylic acid), which binds to avidin with a K_{D} of 7×10⁻⁶M and dissociates in the presence of biotin with a change in absorbance that can be measured spectrophotometrically [59].

Determination of off-rates—To determine differences in the off-rates for the different biotin conjugates described in Table 1, solutions will be added to well plates with covalently immobilized avidin or streptavidin (Life Technologies). After one hour of incubation, a 100-fold molar excess of free biotin will be added, and samples will be collected periodically over 24 hrs [60]. Concentrations of released biotinylated protein will be measured by ELISA, and K_{off} is the slope the fraction of the sample remaining bound to the well plates over time [60]. The presence of biotin conjugated to IL4 does not affect its quantitation by ELISA [19]. CAPTAVIDIN™ protein reagent is not commercially available pre-immobilized to well plates, so for comparisons between avidin, streptavidin, and CAPTAVIDIN™ protein reagent, the proteins will be immobilized on high-binding ELISA plates (R&D Systems) overnight prior to experiments. Alternatively, commercially available agarose bead suspensions with these proteins can be used.

SPR—SPR experiments will be conducted using the methods used routinely in the lab of Dr. Irwin Chaiken, by immobilizing avidin proteins on the surface using standard

EDC/NHS chemistry, which does not affect biotin binding [60]. Biotin conjugates will be flowed over the surfaces at 50-100 μl/min and the change in signal will be measured over time to calculate on-rate. The off-rates will be measured by switching the flow media to fresh buffer.

Molecular simulations—All-atom explicit solvent molecular dynamics techniques will be used to model the interactions between biotin conjugates and avidin proteins using similar techniques as those described by Drs. Chaiken and Abrams in [61].

Statistical analysis—All experiments will be conducted in quadruplicate. On- and off-rates will be compared in each experiment in Table 1 using one way ANOVA and Tukey's post hoc analysis.

Results—Conjugation of proteins to biotin increases steric hindrance and results in accelerated dissociation from avidin and avidin-like proteins, to result in equilibrium constants (K_{D}) ranging from 10⁻⁶ to 10⁻¹⁵M (the lower and upper bounds corresponding to HABA and free biotin). The biotin conjugate/biotin-binding protein pair with the fastest dissociation is CAPTAVIDIN™ protein reagent and desthiobiotin-IL4 with the shortest spacer arm, and the slowest dissociation is streptavidin with biotin-IL4 with the longest spacer arm.

Alternative Strategies - Molecular simulations will be used to probe any unexpected findings. The avidin protein can be further mutated by changing amino acids near the biotin-binding site in order to increase dissociation, which has been thoroughly described in the literature [9]. Also, although a major goal is to employ commercially available biotinylation reagents so that the methods can be easily adapted by other groups, other ligands besides biotin can be used to further control release, including HABA or synthetic peptides that bind to avidin with high specificity and tunable dissociation constants [62].

Impact and significance—The results provide fundamental insight into the changes in biotin-avidin interactions upon conjugation to proteins, a ubiquitous tool in biotechnology, through systematic experimental variation of bioconjugation parameters. The use of entirely commercially available reagents ensures widespread adoption of the techniques to suit various applications ranging from imaging to nanoassembly to biomaterials fabrication.

### Example 4

### Release of Biotinylated Protein from Engineered Tissue

The amount of biotin that can be conjugated to engineered cartilage without affecting its viability is tested. Cartilage tissue is engineered using human mesenchymal stem cells (MSCs) in porous collagen scaffolds as previously described [27, 65] with weekly assessment of cartilage growth by standard immunohistochemical and biochemical assays, including increasing presence of proteoglycans and type II collagen. After 4 weeks of cultivation, cartilage constructs will be biotinylated by incubation with EZ-LINK® protein reagents (ThermoFisher Scientific). Increasing amounts of biotin will be conjugated to the cartilage tissue by increasing the molar excess of reagent, and the effects on tissue viability will be measured using viability assays (eg. MTT) and histology to visualize structural changes. The amount of biotin bound to the cartilage tissue, and its depth of penetration, will be measured using fluorescent streptavidin and anti-biotin antibodies, visualized by confocal microscopy. In this way the depth of biotin penetration can be used to further control the release of proteins via the diffusion term in Equation 1.

Cartilage constructs together with IL4 will be immersed in growth media for additional weeks or months as necessary to completely characterize the release of IL4. The release of IL4 or of avidin is not expected to affect cartilage growth [66, 67], and these findings are confirmed by measuring biochemical content of the cartilage tissue compared to controls treated in the same way but without IL4 or avidin.

Collagen scaffold preparation—Discs are cored from sheets of 2 mm thick Avitene Ultrafoam collagen sponges using 4 mm biopsy punches and lightly crosslinked with standard EDC/NHS chemistry (0.05%), which has only a slight effect on macrophage phenotype in vitro, in order to limit scaffold dissolution [Witherel et al.]. Thus, release of IL4 will depend on diffusion and biotin-avidin interactions and not scaffold degradation or dissolution.

Cultivation and characterization of engineered cartilage constructs—Cartilage constructs will be engineered in vitro using previously described techniques [27, 65, 69, 70]. Briefly, bone marrow-derived human MSCs will be obtained from a commercially available source (eg. Lonza) and expanded to passage 3 in expansion media (high glucose DMEM with 10% FBS). Porous collagen scaffolds will be seeded with 20×10⁶ million MSCs and cultured in chondrogenic serum-free media (high glucose DMEM with 5 mg/ml proline, 1% ITS, 100 nM dexamethasone, 50 μg/ml ascorbate, and 100 ng/ml TGFI33) for the first 2 weeks followed by expansion media. Constructs will be characterized after 0, 2, and 4 weeks for markers of the cartilage phenotype (please see Table 3).

Release studies—Scaffolds and tissues will be biotinylated as described above, joined with IL4 using the selected avidin protein, and immersed in PBS or cartilage growth media at 37° C. Samples of release media will be collected every day or at every media change and replaced with fresh media for several months or until there is no longer a detectable amount of IL4, measured using ELISA. At the end of the release study, scaffolds will be degraded in collagenase to determine the concentration of any residual biotinylated protein that was not released. Bioactivity of released IL4 will be confirmed by adding at 40 ng/ml to human macrophages cultured and characterized as described.

Statistical analysis—All experiments will be conducted with n=6 replicates, which yields significant results with sufficient power [19, 20]. Differences in release kinetics will be determined using a two way ANOVA for the effects of biotin/avidin pair and time.

Results—The formulations evaluated in Table 1 result in a range of release profiles ranging from slow (several months) to medium (several weeks) and fast (several days) release of biotinylated IL4.

Alternative Strategies—As shown in Equation 1, release can be further tuned by varying the starting concentrations of biotin and avidin as well as the depth of tissue penetration to control diffusion. If necessary, release can be made faster by the use of mutated avidin proteins that show reduced binding affinity for biotin [9], or it can be lengthened by hindering diffusion through the use of a coating (porous scaffolds) or by increasing the depth of tissue biotinylation based on time of incubation. Because biotin can be conjugated to cells without damaging their viability [71], there are no major effects on cartilage viability, but the degree of biotinylation will be limited to the minimum amount necessary for control over release.

Impact and significance: The results provide evidence that any biomaterial or engineered tissue can be converted into a controlled release system using biotin-avidin interactions. This technique is applicable to a diverse array of applications in biomaterials and tissue engineering.

### Example 5

### Effects of Polarized Macrophage-Derived Signals on Cartilage Tissue

To evaluate the effects of polarized macrophages on engineered cartilage, cartilage constructs will be cultivated in chondrogenic media mixed in a 1:1 ratio with media conditioned by M0, M1, M2a, or M2c macrophages for 4 weeks. Constructs will be characterized each week for changes in biochemical content and expression of genes associated with chondrogenesis, chondrocyte hypertrophy, and osteogenesis (Table 3). Biochemical and gene expression markers of cartilage will be highest in M2a-conditioned media, while M1 macrophages will promote hypertrophy and ossification and M2c macrophages will promote tissue breakdown. Engineered cartilage can also be co-cultured directly with polarized macrophages instead of using conditioned media if crosstalk is necessary to evaluate effects.

TABLE 3
Markers of chondrogenesis, hypertrophy, and osteogenesis.

| | | Hypertrophic | |
|---|---|---|---|
| | Cartilage | cartilage | Bone |
| Gene | Col2a1, Acan, | Col10a1, Ctgf | Col1a1, ALPL, |
| expression | Sox9, COMP | MMP13, Runx2, | SPP1, OCN, OPN, |
| (RTPCR) | | VEGF | BMP2, Runx2 |
| Immunohisto- | Type II | Type X collagen | Type I collagen |
| chemistry | collagen | | |
| Histology | Proteglycans | Enlarged | Calcium phosphate |
| | (Safranin'O), | chondrocytes | deposits (Von kossa, |
| | Collagen | (hematoxylin & | Alizarin red) |
| | deposition | eosin, H&E) | |
| | (Masson's | | |
| | trichrome) | | |
| Biochemical | Proteoglycans | N/A | Alkaline |
| assays | (DMMB), | | phosphatase (ALP) |
| | collagen | | activity, |
| | (OHP assay) | | mineralization (von |
| | | | kossa, Alizarin red) |

### Example 6

### Control Over Macrophage Behavior In Vivo

The effects of IL4 release on the inflammatory response to engineered cartilage tissue in vivo will be evaluated in a preliminary study utilizing the subcutaneous implantation model in athymic C57BL/6 mice. Athymic mice are unable to mount an adaptive immune response, so human tissues can be implanted without rejection, but the innate immune system (including macrophages and the inflammatory response) remains intact [72]. The subcutaneous model is a simple yet robust way to measure the inflammatory response to engineered tissues in the absence of confounding effects of the intended tissue environment, including mechanical loading and other tissue-specific cell types. The results can then be applied to a large animal model of arthritis.

Four groups will be evaluated: cartilage constructs without IL4 (negative control), constructs that release IL4 over a few days (fast-releasing) or a few weeks (slow-releasing), and constructs with covalently conjugated IL4 (control for the effects of immobilized IL4). Macrophage phenotype surrounding the constructs will be measured with immunohistochemical analysis. The effects on cartilage growth will be assessed as described in Table 3. The release of IL4 will result in M2a polarization and increased quality of cartilage tissue (proteoglycan and collagen content, decreased hypertrophy and ossification) compared to the negative control. Furthermore, the optimal release profile of IL4 will be several weeks, which is slow enough to ensure control over macrophage behavior for the duration of the initial inflammatory response, but fast enough to ensure efficient macrophage polarization.

Macrophage culture and polarization—Polarized macrophages will be prepared from peripheral blood-derived monocytes as in previous studies in which have been extensively characterized their phenotype [20, 37, 46]. Briefly, a 7-day differentiation and polarization protocol is used to prepare M1 (100 ng/ml interferon-gamma (IFNg)+100 ng/ml lipopolysaccharide (LPS)), M2a (40 ng/ml IL4+20 ng/ml IL13), or M2c (40 ng/ml) macrophages, plus a relatively unactivated population (M0) cultured in the absence of polarizing stimuli. Gene expression of a panel of macrophage phenotype markers is always used to confirm robust polarization [20, 37]. Markers of the M1 phenotype include CCR7, TNFa, IL1b, CD80, and VEGFA; markers of the M2a phenotype include CCL18, CCL22, MRC1, PDGFB, and TIMP3. Markers of the M2c phenotype include CD163, MMP7, MMP8, MMP9, VCAN, and MARCO. Conditioned media is prepared by replacing the media with fresh media containing no cytokines for an additional 24 hrs, resulting in the generation of media containing macrophage-derived signals but without the potentially confounding effects of the polarizing cytokines [20].

Subcutaneous implantation model and methods of characterization—Surgeries and veterinary care will be conducted according to the Guide for the Care and Use of Laboratory Animals and protocols approved by the Institutional Animal Care and Use Committee (IACUC) at Drexel University, including procedures for limiting discomfort, distress, and pain [73]. Animals will be anesthetized during surgery with 2.5% isoflurane and euthanized at the conclusion of the experiments by overdose of isoflurane, in accordance with the AVMA Guidelines on Euthanasia. Subcutaneous implantation of the scaffolds in the dorsum of each mouse will be conducted as in previous studies, using a single incision, the creation of space for the scaffolds using forceps, and a suture clip to close the incision [20, 27, 37]. One scaffold from each of the four groups (engineered cartilage with no cytokines, fast-releasing IL4, slow-releasing IL4, or covalent IL4) will be implanted into the subcutaneous space in the dorsum of one mouse (n=6 total). 4 scaffolds can be implanted into one mouse without conflicting effects on macrophage behavior [19] or cartilage growth [27]. After 21 days of in vivo cultivation, cartilage constructs together with any fibrous capsule will be excised and cut in half. Half of each construct will be used for biochemical and gene expression analysis, and the other half of each construct will be fixed in 4% paraformaldehyde overnight and embedded in paraffin for histological and immunohistochemical analysis (Table 3). Samples will be sectioned and stained with triple immunofluorescence for markers of the M1, M2a, and M2c phenotypes [20, 37]. Briefly, serial sections will be stained for the pan-macrophage marker F4/80 in combination with either iNOS and Arg1 (to distinguish between M1 and M2a) or Arg1 and CD163 (to distinguish between M2a and M2c). The intensity of expression of each marker will be used as an indicator of macrophage phenotype, which is more indicative of a specific phenotype that simply counting the number of cells staining positively for a given marker [20, 37].

Statistical analysis—The effects of IL4 release profile on each parameter describing cartilage behavior will be compared using a one way ANOVA with Tukey's post hoc analysis.

Results—M1 and M2c phenotypes of macrophages will hinder cartilage growth. M1 macrophages also promote cartilage hypertrophy and ossification based on previous studies showing the osteogenic effects of M1 macrophages [24]. In contrast, because M2a macrophages secrete multiple factors that promote cartilage growth, these macrophages support cartilage growth in vitro and in vivo.

Alternative Strategies—If it is determined that the injury created by the implantation causes such a substantial inflammatory response that the release of IL4 is not sufficient to induce M2a polarization, release of XPro1595, a specific inhibitor of pro-inflammatory tumor necrosis factor-alpha (TNFa) [74] in addition to IL4, will inhibit M1 and induce M2a polarization. It is also possible that the M2c phenotype will be beneficial for cartilage growth, alone or in combination with the M2a phenotype, based on the fact that IL10 has been used in vivo for the treatment of arthritis [25] and that tissue breakdown can be a healthy component of remodeling and regeneration in many tissues, provided that it is balanced with tissue growth. This idea will be tested by culturing cartilage tissue in media containing signals from both M2a and M2c macrophages, and constructs to be implanted in vivo can be prepared to release IL10 in combination with IL4.

Impact and significance—The results provide a fundamental understanding of how interactions with macrophages of multiple phenotypes affect engineered tissue, and proof-of-concept that the in vivo manipulation of host macrophage phenotype can be used to enhance tissue engineering strategies. These results pave the way for strategies to harness, rather than inhibit, the inflammatory response in regenerative medicine.

### Example 7

### Neuronal Regeneration

Drug delivery systems that shift macrophage behavior from a pro-inflammatory (aka M1) to a pro-regenerative (aka M2c) phenotype mitigate neuronal scarring, promote neuronal recovery and enhance circuit regeneration using the body's natural healing mechanisms.

A. Nanoparticles for Targeting Macrophages Following SCI and TBI

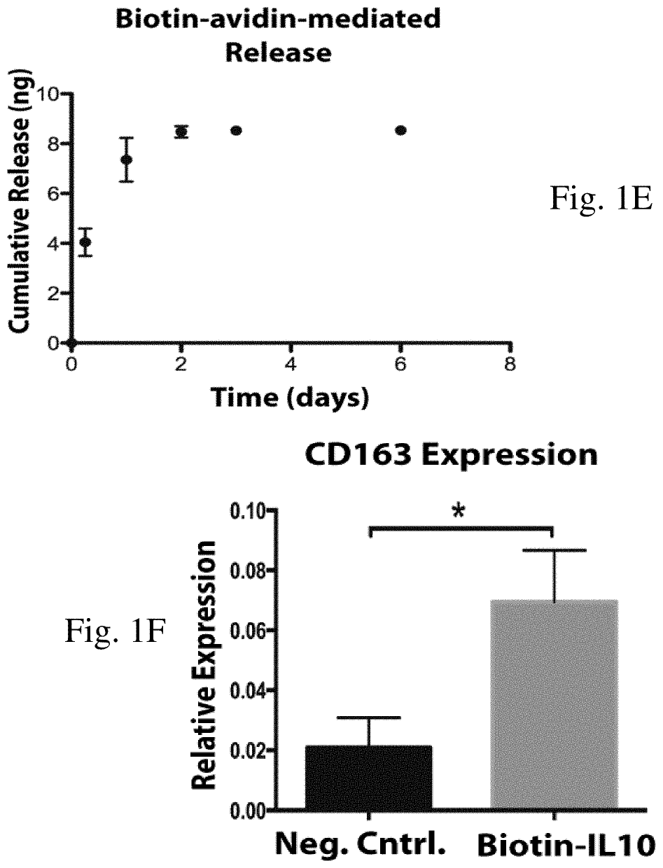

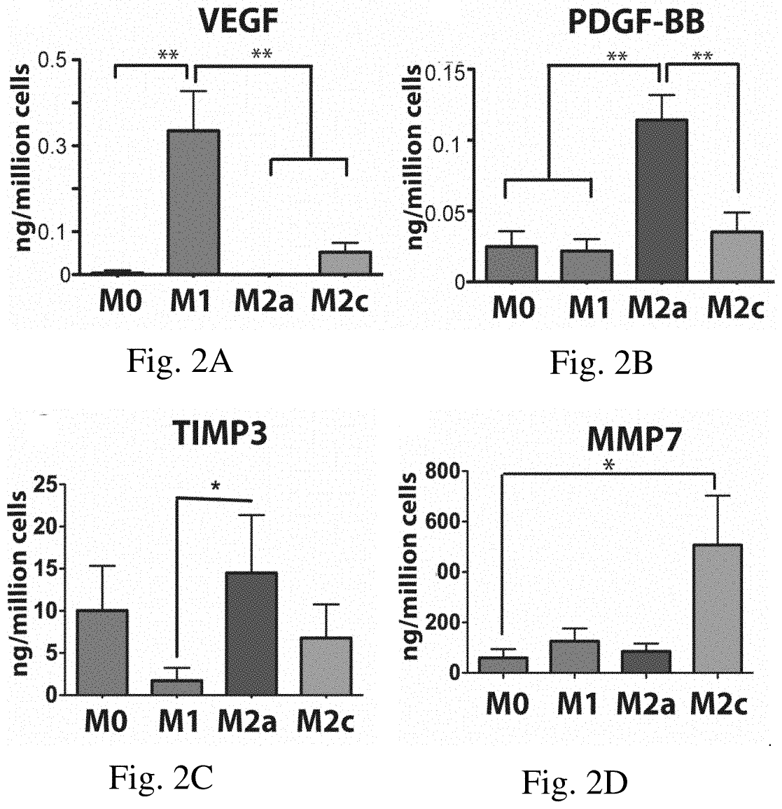

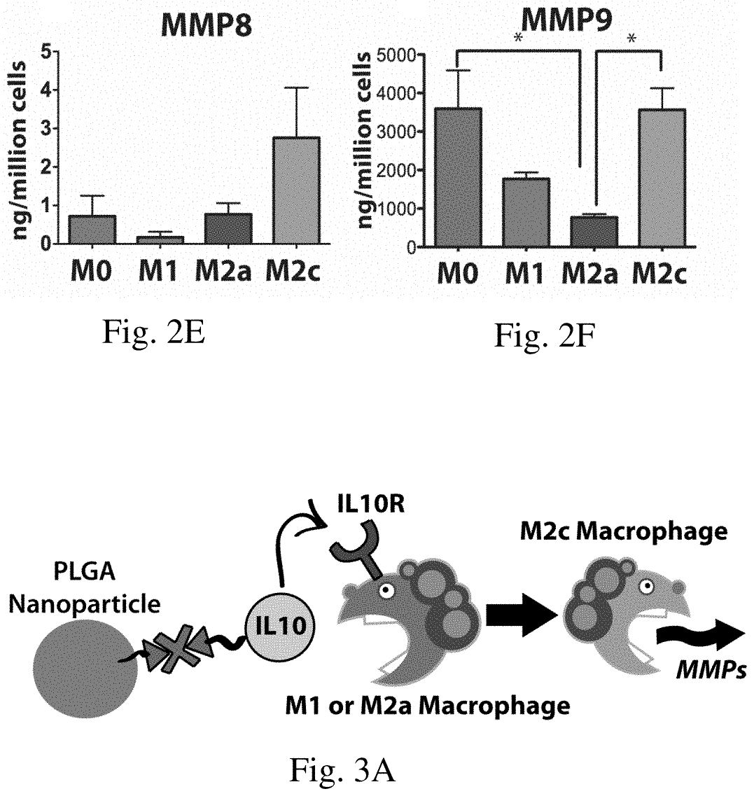

Macrophages preferentially interact with and phagocytose nanoparticles relative to other cell types. Thus, as IL10 on the surface of the nanoparticles comes into contact with the IL10 receptor on the surface of macrophages, it is released from the nanoparticles because the affinity between IL10 and its receptor is greater than that for conjugated biotin with avidin, causing polarization of the macrophages to the pro-regenerative phenotype (FIG. 3A). These polarized macrophages home to the site of injury and release pro-regenerative factors as well as scar- and inhibitory molecule-degrading enzymes like matrix metalloproteases (MMPs).

B. Modulating the Inflammatory Response to Engineered Nerve Grafts

Cullen [80] has pioneered the engineering of nerve grafts consisting of living neurons and axonal tracts embedded within hydrogels for the treatment of central nervous system disorders and traumatic brain injury. However the inflammatory response and foreign body response to the implanted constructs threaten their long-term viability. These nerve grafts can be biotinylated without affecting their viability, allowing the controlled release of macrophage-modulating factors like IL10, promoting graft incorporation and enhancing neuronal circuitry coupling.

### Example 8

### Scaffold Preparation

A. Methods

Isolation and Culture of Primary Human Macrophages

Monocytes were isolated from enriched leukocyte fractions of human peripheral blood purchased from the New York Blood Center using sequential Ficoll and Percoll density gradient centrifugations. Monocytes were cultured at 37° C. and 5% CO₂ in ultra low attachment flasks (Corning) for five days at a density of 0.4×10⁶ cells/cm² and 1.0×10⁶ cells/ml of complete media (RPMI media supplemented with 10% heat-inactivated human serum, 1% penicillin-streptomycin, and 20 ng/ml macrophage colony stimulating factor (MCSF)). Macrophages were polarized over the next 1-6 days by culturing at 1.0×10⁶ cells/ml in complete media with 100 ng/ml IFN-gamma (Peprotech, Rocky Hill, N.J.) and 100 ng/ml lipopolysaccharide (LPS, Sigma Aldrich) for M1 or 40 ng/ml IL4 and 20 ng/ml IL13 (Peprotech, Rocky Hill, N.J.) for M2, with a media change at day 3. At the media change, the media of another group of M1 macrophages was switched to M2-polarizing media and the media of a group of M2 macrophages was switched to M1-polarizing stimuli, in order to characterize the ability of macrophages to switch phenotypes. Unactivated macrophages were also cultured over the same time periods (M0), resulting in three groups through day 3 (M0, M1, M2) and five groups between days 4 and 6 (M0, M1, M2, M1→M2, M2→M1).

Characterization of Macrophage Phenotype

At days 1, 2, 3, 4, and 6, macrophages were collected by gentle scraping and centrifugation. The number of viable cells was determined at each time point by trypan blue exclusion. Macrophages from each time point were characterized for expression of known M1 and M2 markers by quantitative RT-PCR. For flow cytometry, cells were dual-stained with APC-conjugated CCR7 (Biolegend.com, catalog no. 353213, dilution 1:50) and FITC-conjugated CD206 (Biolegend.com, catalog no. 321103, dilution 1:100). Corresponding isotype controls were used as recommended by the manufacturer. Labeled cells were analyzed using a FACSCALIBUR™ flow cytometer and the CELLQUEST™ software (both from BD Biosciences, Pharminogen). Data was processed using FLOWJO® software (Treestar). To determine the proportion of cells staining for a given marker at high or low levels, the mean intensity of staining of the M0 population was used as a threshold. In other words, cells staining more intensely for CCR7 than the mean of the M0 population were considered CCR7^{hi}, while those staining less intensely than the mean of the M0 population were considered CCR7^{lo}. This analysis was performed similarly for CD206^{hi} and CD206^{lo} populations, allowing determination of the proportion of cells that were both CCR7^{hi} and CD206^{lo} and those that were both CCR7^{lo} and CD206^{hi}.

At each time point, the supernatant was frozen at −80° C. until analysis by enzyme-linked immunosorbent assays (ELISA). Secreted M1 markers included tumor necrosis factor-alpha (TNF-alpha) and VEGF (Peprotech) and M2 markers included CCL18 (R&D Systems) and PDGF-BB (Peprotech).

Preparation and Biotinylation of Scaffolds

Decellularized bone scaffolds were prepared from trabecular bone by coring plugs from the subchondral regions of young cows and washing with water and detergents. Scaffolds (4 mm in diameter and 2-3 mm in height) were separated based on density, calculated by measuring the height, diameter, and mass of cylindrical samples, in order to ensure uniformity between experiments. The average density of the scaffolds used in this study was 0.49±0.03mg/mm³ (mean±standard deviation).

Scaffolds were sterilized by soaking in 70% ethanol for 24 hours, followed by washing in phosphate-buffered saline (PBS). Then, scaffolds were biotinylated using NHS (N-Hydroxysuccinimide) chemistry by immersion in 10 mM Biotin-sulfo-LC-LC-NHS (EZ LINK® protein reagent, Thermo Fisher Scientific, Rockford, Ill.) for one hour, followed by three washes with 2 ml PBS to remove unattached biotin. Scaffolds were briefly immersed again in 70% ethanol for 10 min, followed by three more washes, and finally immersed in PBS at 4° C. overnight prior to attachment of biotinylated proteins.

The extent of scaffold biotinylation was determined after mixing with avidin and HABA (4′-hydroxyazobenzene-2-carboxylic acid, Thermo Fisher Scientific, Rockford, Ill.). HABA binds strongly to avidin, but is displaced by biotin, which binds at a much higher affinity, causing a decrease in the absorbance of HABA, which can be read spectrophotometrically. A standard curve of biotin was prepared in a 96-well plate using non-biotinylated scaffolds together with 20 ul of biotin solutions ranging from 0 to 100 ug/ml. 180 ul of a solution of HABA and avidin (2.69 mg/ml HABA and 0.467 mg/ml avidin) was added to each well containing the standards or the biotinylated scaffolds. After 1 minute the scaffolds were removed and the absorbance was read at 500 nm. The difference in absorbance from blank controls was used to generate a standard curve and to calculate the amount of biotin on each scaffold.

In preliminary studies, an approximately 50-fold excess of biotin to protein content of the scaffolds (calculated using the assumption that the protein was 100% collagen) was found to result in the same level of biotinylation as up to 500-fold molar excess. Therefore a 50-fold molar excess of biotin was used for scaffold biotinylation.

Protein Biotinylation and Conjugation to Scaffolds

IL4 was biotinylated by adding a 100-fold molar excess of the 10 mM Biotin-sulfo-LS-LS-NHS for one hour, followed by dialysis overnight to remove unattached biotin, and then sterile-filtered. Retention of bioactivity was 75%, determined using an IL4 ELISA (Peprotech). Four groups of scaffolds were prepared: scaffolds with attached IL4 (IL4), scaffolds with adsorbed IFNg (IFNg), their combination (Combo), and a negative control (Neg. Cntrl.), which was prepared in the same way as the other scaffolds but using PBS instead of IFNg or IL4 solutions.

For all groups, biotinylated scaffolds were soaked in 0.5 ml of 172 μg/ml streptavidin (Thermo Fisher Scientific) for 1 hour, followed by washing 3 times in PBS. To prepare the IL4 and Combo groups, scaffolds were soaked in 375 ng biotinylated IL4 in 0.5 ml of PBS for 1 hour, while Neg. Cntrl. and IFNg groups were soaked in PBS. Streptavidin has four binding sites for biotin with extremely high specificity and strength, creating a strong but not covalent linkage between IL4 and the scaffolds. To determine that streptavidin bound specifically to biotin on the scaffolds, biotinylated scaffolds were incubated with fluorescent STREPTAVIDIN-DYLIGHT-594® secondary antibody (Thermo Fisher Scientific) and compared to non-biotinylated scaffolds using confocal laser scanning microscopy. Following streptavidin binding, scaffolds were washed 3 times with 2 ml PBS to remove unattached IL4. Then, scaffolds in the IFNg and Combo groups were incubated in IFNg (325 ng/scaffold) for 1 hour to allow physical adsorption, while Neg. Cntrl. and IL4 scaffolds were soaked in PBS. Scaffolds were then transferred to 24-well ultra low attachment plates for release studies or for macrophage culture.

Characterization of Release Profiles The amount of bound IFNg and IL4 on each scaffold was assessed indirectly by measuring the amount of protein in the wash solutions using ELISA kits (Peprotech). To characterize the release of IFNg and IL4 proteins from the scaffolds, scaffolds from each of the four groups were incubated in 1 ml complete media for 11 days at 37° C. and 5% CO₂, with samples taken and media refreshed at 6 hrs, 1 day, 2 days, 3 days, 6 days, and 11 days. The amount of IFN-gamma and IL4 in each sample was determined using ELISA (Peprotech). Values obtained for the negative control scaffolds were subtracted from the experimental groups at each time point. Samples of biotinylated IL4 were also assayed in both IFNg and IL4 ELISAs to ensure that there was no nonspecific binding.

Macrophage Seeding and Characterization

Macrophages were collected 5 days after differentiation from monocytes and seeded onto the scaffolds at 8.0×10⁵ per scaffold in 20 μl of complete media (n=6). The cells were allowed to attach for 1 hour before the addition of 1 ml complete media. The cell-seeded constructs were cultured for 3 and 6 days, with a media change after 3 days. The media was frozen at −80° C. until analysis for M1 and M2 markers by ELISA, as described above. To extract RNA from the scaffolds, the scaffolds were immersed in 1 ml TRIZOL® chemical reagent for isolating RNA (Life Technologies) with 5-6 steel beads (0.5 mm diameter) and homogenized for 6 cycles of 10 seconds in a Mini BEAD-BEATER™ homogenizer (Biospec Products, Bartlesville, Okla.). RNA was extracted into chloroform, which was then purified using an RNEASY® Micro Kit chromatographic material (Qiagen) according to the manufacturer's instructions. DNase treatment, cDNA synthesis, and RT-PCR was performed.

LPS Contamination

Cell culture media was periodically tested for contamination with LPS using the LAL Chromogenic Endotoxin Quantification kit (Thermo Scientific Fisher) per the manufacturer's instructions. LPS contamination was always below 0.1 EU/ml.

Subcutaneous Implantation Model

All animal experiments followed federal guidelines and were conducted under a protocol approved by Drexel University's Institutional Animal Care and Use Committee. Scaffolds were prepared as described above except using murine cytokines (Peprotech). One scaffold from each of the four groups was implanted subcutaneously in female 8-week-old C57BL/6 mice for two weeks (n=3 mice). Mice received a subcutaneous injection of buprenorphine (0.1 mg/ml) for pain, anesthetized using isofluorane (1-5%), shaved, cleaned with ethanol and iodine, and then draped for surgery. A small incision was made in the central dorsal surface using a scalpel. Blunt forceps were used to create a pocket in the subcutaneous space for the scaffolds. After implantation, wounds were closed with one wound clip. Mice were housed together and monitored for 14 days. No signs of pain or discomfort were observed following surgery or throughout the study.

Following 2 weeks of in vivo cultivation, mice were euthanized by CO₂ asphyxiation. Scaffolds were explanted, fixed overnight in 4% paraformaldehyde, decalcified in formic acid (IMMUNOCAL™ chemical reagents, Decal Chemical Corporation, Tallman, N.Y.), dehydrated through an ethanol series and embedded in paraffin. Samples were sectioned to 5 μm and stained for general structure using hematoxylin and eosin (H&E). Endothelial cells were visualized via immunohistochemical staining for CD31. Sections were subjected to antigen retrieval by immersion in 95° C. citrate buffer for 20 min, then blocked for 1 hr in 5% bovine serum albumin, then incubated overnight with goat-anti-mouse CD31 (dilution 1:30, Santa Cruz Biotechnology, catalog no. sc-1506) and visualized using a donkey-anti-goat secondary antibody conjugated to FITC (Santa Cruz Biotechnology, catalog no. sc-2024), counterstained with DAPI (Vector Labs DAPI mounting medium). Fluorescent images of CD31 staining were acquired on an Evos Fl Digital inverted fluorescence microscope. The number of CD31+ vessels within the samples was quantified in at least six images per section (10× magnification) and two sections per sample using ImageJ. The mean fluorescence intensity of the delete primary negative control was subtracted from that of the samples. Samples were also analyzed for macrophage phenotype markers. Samples were triple-stained for the M1 marker iNOS, the M2 marker Argl, and the pan-macrophage marker F480. At least six images per section (20× images) and two sections per sample will acquired on an EVOS™ FL digital inverted fluorescence microscope (Thermo Fisher Scientific). The mean intensity of expression of each marker in the cellular portion of the images was quantified in ImageJ. Intensity was analyzed as opposed to counting cells because intensity is a better marker of macrophage phenotype than the presence or absence of a marker. The mean intensity of expression in the delete primary control images was subtracted from the value obtained each marker to account for nonspecific staining.

Samples were also analyzed for the presence of IL4 using rabbit-anti-mouse IL4 (1:10 Thermo Scientific Pierce, catalog no. PA 525165) and goat-anti-rabbit secondary antibody conjugated to DyLight488 (Thermo Scientific Pierce).

Statistical Analysis

Data are represented as mean ±SEM. Data from all in vitro experiments are representative from one of at least three repeated experiments. Statistical analysis was performed in GraphPad Prism 4.0 using one-way ANOVA and either Tukey's or Dunnett's post-hoc analysis, as indicated. A p-value of less than 0.05 was considered significant.

B. Results

Kinetics of Macrophage Phenotype Switching

Over 6 days of culture in polarizing stimuli, M1 and M2 macrophages gradually increased surface marker expression of CCR7 and CD206, with M1 macrophages staining more strongly for CCR7 and M2 macrophages staining more strongly for CD206. When M1 macrophages were given M2-promoting stimuli at day 3, the entire population shifted to express less CCR7 and more CD206. Similarly, M2 macrophages that were given M1-promoting stimuli at day 3 showed reduced CD206 expression and increased CCR7 expression.

Maximum staining was observed at day 4 in terms of both the percentage of the population staining positively. Mean intensity per cell, evaluated, is a better indicator of macrophage phenotype than the percent of cells staining positively. To more accurately describe the change in the numbers of cells representing the M1 and M2 populations, the results gated based on the mean intensities of CD206 and CCR7 expression of the M0 population at the same time point, in order to determine the number of cells that could be described as CCR7^{hi}CD206^{lo}, which would indicate the M1 phenotype, and those that were CCR7^{lo}CD206^{hi}, which would be more indicative of the M2 phenotype. Interestingly, the greatest changes in expression were seen at day 4, or one day after the media change at day 3, even for control phenotypes that were not switched, indicating that the macrophages were able to respond to increased stimulus. In addition, the change from M1→M2 appeared more dramatic than the change from M2→M1, in that the latter group did not show expression of CCR7 after 6 days at the same levels as M1 controls, even though M1→M2 cells showed levels of CD206 that were higher than M2 controls at day 6.

Gene expression of the M1 markers TNFa, ILlb, CCR7, and VEGF was highest for M1 macrophages and increased over time, with the highest expression at day 6. In keeping with flow cytometry results, a dramatic increase was seen at day 4, after the media change. The addition of M2-promoting stimuli at day 3 effectively inhibited expression of these genes and caused upregulation of the M2 markers CCL18, MDC/CCL22, CD206/MRC1, PDGF, and TIMP3. M2 macrophages showed high levels of expression of the M2 markers, with maximum expression at day 3, until the media was changed to M1-polarizing stimuli, at which point they decreased expression of M2 markers and increased expression of M1 markers. Both M1 and M2 macrophages that were switched to the other phenotype expressed genes comparable to or higher than the control phenotypes. Interestingly, however, M1 macrophages that were switched to the M2 phenotype did not down regulate VEGF expression, although they did increase expression of PDGF. Moreover, M2 macrophages that were switched to the M1 phenotype showed equally high expression of both VEGF and PDGF at day 6, indicating a mixed or hybrid phenotype. Control M1 macrophages also increased expression of PDGF at day 6, suggesting that they may naturally increase expression of this gene over time.

M1 macrophages secreted high levels of TNF-alpha and VEGF, with maximum secretion at days 4-6. The addition of M2-polarizing stimuli caused drastic inhibition of secretion of these markers and increased in secretion of the M2 markers CCL18 and PDGF-BB, compared to control M1 macrophages that were stimulated for 6 days.

Similarly, the addition of M1-polarizing stimuli to M2 macrophages caused decreased secretion of M2 markers CCL18 and PDGF-BB as well as increased secretion of the M1 markers TNF-alpha and VEGF.

M2 macrophages, including M1 macrophages that were switched to the M2 phenotype, proliferated over time in culture. When the amount of secreted proteins was normalized to the number of viable cells at each time point, the amounts of M2 markers secreted by M1 macrophages that were switched to M2 media were only slightly higher than the M1 control.

Release of IFNg and IL4

Having confirmed that M1 macrophages can switch to the M2 phenotype, their behavior was examined on scaffolds designed to elicit sequential M1 and M2 polarization. Decellularized bone scaffolds were biotinylated using NHS chemistry. Streptavidin was found to only bind to scaffolds that were biotinylated, with undetectable nonspecific binding to control scaffolds after washing.

Indirect measurement of the content of unbound proteins in the wash solutions used in the preparation of the scaffolds suggested that 26.9±10.3 ng of IFNg and 153.3±48.5 ng of IL4 attached to the scaffolds. However, release studies showed that less than 1 ng of the adsorbed IFN-gamma was released in the first 48 hours, resulting in a concentration of less than 1 ng/ml in the media. Less than 8 ng of biotinylated IL4 was released over 6 days, with no detectable IL4 in the media after that point. It is likely that the indirect measurement method overestimates the actual loading, and further studies are therefore required to confirm this point.

Release profiles of IFNg and of IL4 were not found to be different for Combo scaffolds, which had both IFNg and IL4, compared to the scaffolds with only IFNg or IL4.

Response of Macrophages to Immunomodulatory Scaffolds

Gene expression data indicated that physical adsorption of IFNg to scaffolds with and without attached IL4 caused increased expression of M1 markers after 3 days of culture. This early M1 polarization was achieved despite low levels of protein released in the first three days (less than 1 ng, compared to the dose of 100 ng that is typically used to polarize macrophages to the M1 phenotype). Expression of M1 markers decreased to background levels by day 6, although expression of TNFa and CCR7 did remain significantly higher for Combo scaffolds compared to the negative control. At both 3 and 6 days, expression of M2 markers was significantly higher for macrophages seeded on scaffolds with attached IL4 compared to the negative control. Macrophages seeded on scaffolds in the Combo group also significantly increased gene expression of M2 markers at day 3, but these increases were not significant at day 6. There were some genes that were not regulated as expected: Expression of CD206 was not significantly different between any of the groups at either time point, despite being a well-known marker of IL4-induced M2 activation. In addition, the M2 marker PDGF was upregulated at day 3 by the presence of IFNg, reminiscent of the hybrid phenotypes of M1 switching to M2.

The amounts of secreted proteins associated with the M1 and M2 phenotypes were measured using ELISA to confirm gene expression results. Adsorption of IFNg caused increases in the secretion of the M1 marker TNF-alpha at 3 days compared to the IL4 group (one-way ANOVA with Tukey's post-hoc analysis, p<0.05). No differences were seen in M1 marker secretion at 6 days. Attachment of IL4, without adsorbed IFNg, caused significant increases in secretion of the M2 marker CCL18, which was sustained at 6 days (one-way ANOVA with Tukey's post-hoc analysis, p<0.001). Attachment of IL4 also increased secretion of PDGF-BB at 6 days compared to the negative control (one-way ANOVA with Dunnett's post-hoc analysis, p<0.05). Interestingly, macrophages seeded on the Combo scaffolds did not show significantly different levels of secretion of any marker compared to the control, despite their ability to promote changes in both M1 and M2 gene expression.

Vascularization In Vivo

After 2 weeks of in vivo implantation, all scaffolds were fully infiltrated by cells. Large blood vessel-like structures were apparent in the IFNg, IL4, and Combo groups, but not in the negative control scaffolds. The endothelial cell marker CD31 was most abundant in IFNg and Combo samples. There were significantly more CD31+ blood vessels in the IFNg scaffolds compared to the negative control scaffolds (p<0.05).

No differences were observed in macrophage phenotype, as indicated by staining for the M1 marker iNOS, the M2 marker Argl, and the pan-macrophage marker F480. Quantification of the mean intensity of each marker in the cellular portion of the scaffolds confirmed that there were no significant differences in staining between groups. Normalization to F480, which would represent amount of staining per macrophage, or normalization of Argl staining to iNOS staining, a relative measure of M2 vs. M1 polarization, also yielded no significant differences.

Murine IL4 was detected in all of the samples, without differences in staining between the groups, indicating that no scaffold-derived IL4 remained after 2 weeks in vivo.

### Example 9

### M2c Phenotype

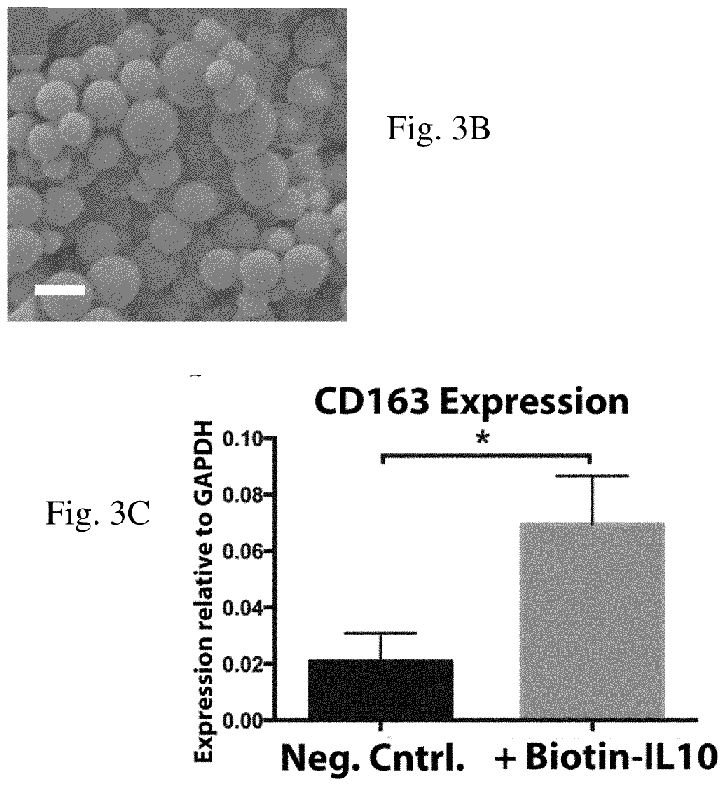

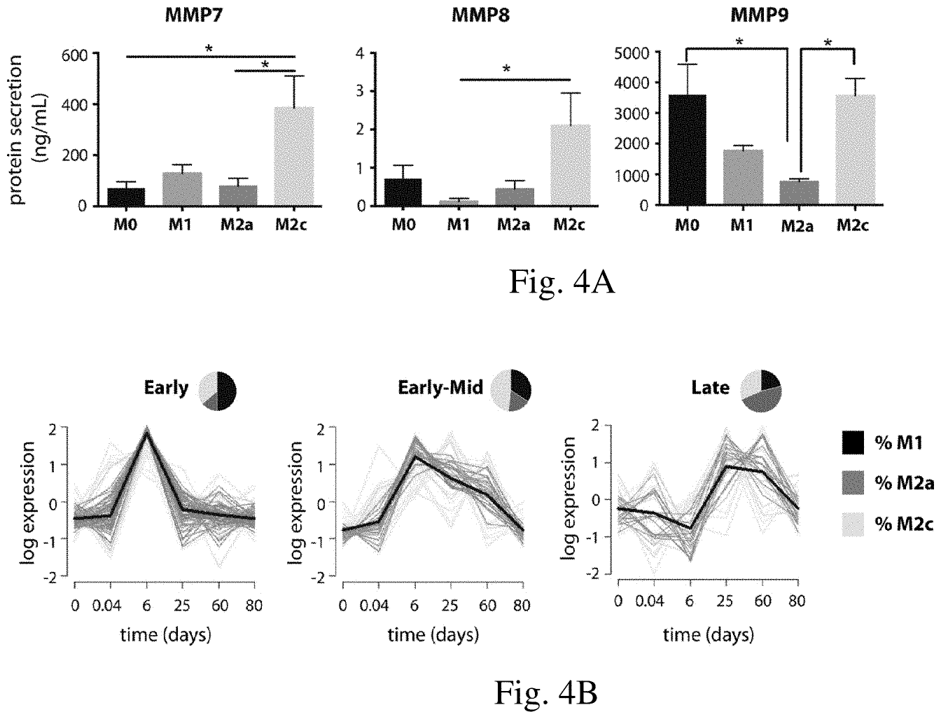

M2c macrophages (those stimulated with IL10) have not been well investigated, perhaps because of a lack of known markers specific to the M2c phenotype, making them difficult to characterize or track in vivo. Next generation sequencing of macrophages polarized in vitro to the M1, M2a, and M2c phenotypes was performed. 19 specific markers of the M2c phenotype were identified and validation, allowing investigation of the M2c phenotype in vivo. Many of the most highly upregulated M2c-specific genes were matrix metalloproteases (MMPs) and other genes associated with matrix degradation (confirmed on the protein level in FIG. 4a), supporting their ability to inhibit fibrosis and to cause branching of nascent blood vessels. Using clustering analysis of three publicly available microarray data sets of human wound healing, it was found that most of the genes that were upregulated at early times after injury were associated with the M1 phenotype, while genes upregulated at later times were primarily M2a markers (FIG. 4b), in agreement with many studies that have described sequential M1 and M2a activation following injury. Interestingly, genes associated with the M2c phenotype were primarily upregulated at early times after injury, in agreement with studies that have shown that M2c macrophages initiate angiogenesis and engulf apoptotic cells, both critical processes in the early stages of wound healing. These results, in combination with others that have associated M2c macrophages with biomaterial vascularization and integration and the fact that IL10 is well-known as an anti-inflammatory cytokine that inhibits M1 activation, suggest that biomaterials that actively promote the M2c phenotype will support vascularization and healing with diminished fibrous encapsulation. In addition, these findings further demonstrate that temporal control will be critical for design of immunomodulatory biomaterials.

### Example 10

### Bioconjugation Parameters Impact on Biotin Dissociation from Avidin

Despite the emergence of commercially available derivatives of biotin and avidin with a range of binding affinities, detailed investigations into the interactions between the length of the spacer arm and the molecular weight of the biotinylated molecule have not been performed. Fluorescent dextran was chosen as a model molecule because it is commercially available in a wide range of molecular weights, with fluorescent tags, and with primary amine groups available for biotinylation using similar methods as those used for proteins. Interactions with avidin proteins will be measured directly through binding experiments and surface plasmon resonance, with further insight gained from in silico investigation.

Effects of Bioconjugation Parameters on Biotin-Avidin Interactions

By measuring the off-rates of all 25 combinations of spacer arm length and conjugate size listed in Table 2, sufficient data is generated to populate a predictive model of dissociation kinetics for other spacer arm lengths and conjugate molecule sizes, determined empirically by nonlinear regression. Then, these results were combined with studies of interactions with derivatives of biotin and avidin that have been mutated to alter binding kinetics, for a more complete analysis of the interactions between these parameters. For example, desthiobiotin is a sulfur-free, single-ring analog of biotin that binds avidin with equal specificity but substantially decreased affinity, increasing the dissociation constant of free biotin from 10⁻¹⁵M to 10⁻¹¹. Nitroavidin is a form of avidin in which the tyrosine residues near the biotin-binding site have been nitrated, increasing the dissociation constant (K_{D}) of free biotin from 10⁻¹⁵M to 10⁻⁶. By comparison, release of a peptide from a protein with a similar K_{D} could be varied from a few days to a few months by changing the ratio of the starting concentrations of peptide to protein. Off-rates of biotin conjugates from avidin are determined from release studies conducted in the presence of a 100-fold molar excess of free biotin, so that release occurs over the time frame of 1-18 hours. All products necessary to prepare the conjugates with manipulations listed in Table 2 are available commercially from Thermo Fisher Scientific, ensuring widespread adoption of the results and adaptation by others to new applications.

SPR and Molecular Simulations

Both the on- and off-rates of the biotin conjugates from avidin proteins are directly measured (in the absence of free biotin) using surface plasmon resonance (SPR). Then the alterations in biotin-avidin binding affinity upon conjugation result from steric hindrance and/or alterations in the thermal stability of the bond are investigated. Existing computational models of avidin-biotin binding are used as the blueprint for designing new biotin conjugates to investigate how conformational interactions are affected by steric hindrance (length of spacer arm, size of conjugate) as well as by structural changes in the binding site (biotin vs. desthiobiotin; avidin vs. nitroavidin). These molecular simulations allow development of predictive models of how bioconjugation parameters affect association/dissociation kinetics, so that the system can be applied to predict the kinetics of other biotin conjugates.

Methods

Dextran Biotinylation

Dextrans with a range of molecular weight and containing fluorescein and primary amines (available from Thermo Fisher Scientific) are biotinylated using the EZ-LINK® protein reagents with varying spacer arm lengths (Thermo Fisher) by incubating with a 20- to 50-fold molar excess of biotin-NHS for 2hrs, according to the manufacturer's instructions. Biotinylated dextran is dialyzed against PBS to remove unreacted biotin. The degree of biotinylation is measured as has been previously described, by adding samples to a solution of avidin bound to the HABA dye (4′-hydroxyazobenzene-2-carboxylic acid), which dissociates in the presence of biotin with a change in absorbance that can be measured spectrophotometrically.

Determination of Off-Rates

Biotinylated dextran is added to well plates with covalently immobilized avidin (Thermo Fisher). After one hour of incubation, a 100-fold molar excess of free biotin is added to displace the biotinylated molecules, which have lower binding affinity, and samples will be collected periodically over 24 hrs. Concentrations of released biotinylated fluorescent dextran will be measured using a fluorescent plate reader to calculate K_{off}, the slope of the fraction of the sample remaining bound to the well plates over time.

SPR—Surface Plasmon Resonance

SPR experiments are conducted using the methods used routinely by immobilizing avidin proteins on the substrate using standard EDC/NHS chemistry, which does not affect biotin binding. Biotin conjugates are flowed over the surfaces at 50-100 μl/min and the change in signal will be measured over time to calculate on-rate, and by switching the flow media to fresh buffer to measure off-rates.

Molecular Simulations

All-atom explicit solvent molecular dynamics techniques are used to model the interactions between biotin conjugates and avidin proteins using similar techniques as those previously described.

Statistical Analysis

All experiments are conducted in quadruplicate. On- and off-rates are compared in each experiment in Table 2 using one way ANOVA and post hoc analysis. Linear or non-linear regression is used to develop an empirical model to predict how binding kinetics change for a given molecular weight or length of spacer arm.

Results and Alternative Strategies

The off-rates of biotin conjugates from avidin increase with increasing molecular weight of the conjugate because of increased steric hindrance and/or decreased thermal stability of the biotin-avidin bond. Similarly, increasing the length of the spacer arm decreases off-rates. Molecular simulations are used to probe any unexpected findings. For parameters described in Table 2 that do not result in appreciable changes in dissociation of biotin from avidin that are required to prepare diverse release profiles, the avidin protein is further mutated by changing amino acids near the biotin-binding site in order to increase dissociation. Other ligands besides biotin are used to further control release, including synthetic peptides that bind to avidin with high specificity and tunable dissociation constants.

With the use of fluorescent dextran (MW 10,000) with pendant lysine groups (as a model protein) was biotinylated through NHS-biotin conjugation, along with porous gelatin scaffolds. Biotinylated scaffolds were incubated in streptavidin for 1 hour. Control scaffolds were prepared with PBS instead of streptavidin. Then biotinylated dextran was added to the scaffolds for 1 hour. Scaffolds were washed 3 times to remove unbound molecules. A 1000-fold molar excess of unconjugated “free” biotin was added to the scaffolds to accelerate release of biotinylated dextran. Fluorescent dextran in solution (released from the scaffolds) was measured after 1 hour. The conjugation of biotin to fluorescent dextran retarded its release from biotinylated scaffolds with bound streptavidin compared to control biotinylated scaffolds with no streptavidin (more dextran was released from control scaffolds after 1 hour compared to scaffolds containing streptavidin). 5400 AU (fluorescence) was observed for control, compared with under 5000 for biotin (FIG. 6).

Fundamental insight into the changes in biotin-avidin interactions upon conjugation to large molecules are provided. These results are critical in developing controlled release systems and for other applications that rely on this technology, such as separation chromatography, imaging, and sensors, among others.

### Example 11

### Impact of Biomaterial Properties on Biotin-Avidin-Mediated Release Kinetics

Release of proteins from complex biomaterials is achieved through manipulations in biotin-avidin dissociation kinetics via biomaterial properties. Release kinetics from affinity-based systems are dictated by the rates of association and dissociation as well as starting concentrations of each reagent, according to the reaction AB⇄A⇄B, such that the rate of change in the concentration of either free species A or B is k_{off}[AB]−kₒₙ[A][B]. Thus, biomaterial properties directly affect the dissociation kinetics by altering the concentrations of the available binding species. For example, biotinylated proteins may accumulate within a hydrogel, driving the reaction toward biotin-avidin association and away from dissociation. Similarly, release kinetics will be different from a porous scaffold, which has many available binding sites for a biotinylated protein compared to a flat biomaterial in which only the surface is modified.

The impact of biomaterial structure and diffusion properties on the release of biotinylated IL10 (the cytokine that promotes the M2c phenotype of macrophages) is characterized over the course of several weeks in vitro. A simplified mathematical model is developed to aid in biomaterial design and allow extension by others to diverse applications. Finally, these principles are used to demonstrate control over release from complex biomaterials including engineered tissues, bioprosthetic heart valves, and titanium implants.

Mathematical Modeling

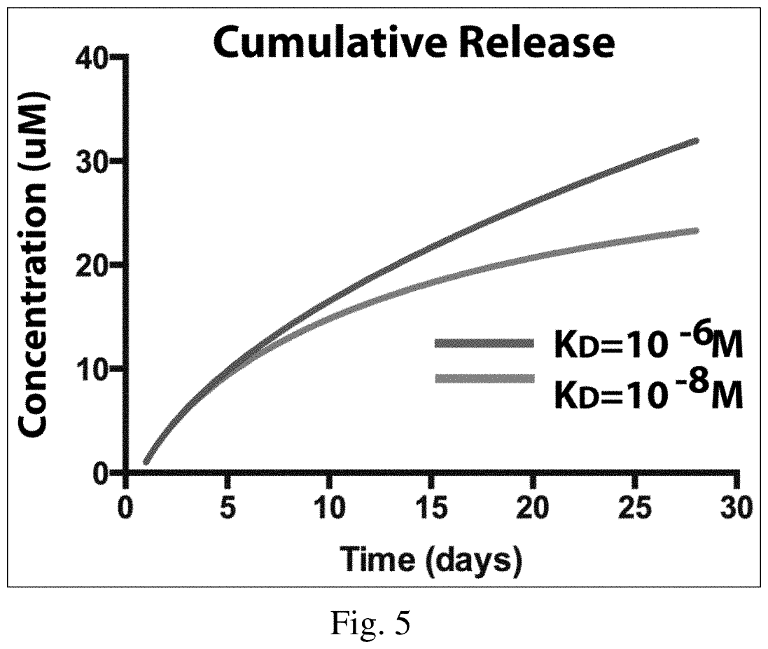

The on- and off-rates of biotinylated IL10 are measured using the techniques described in Example 10 after using the predictive models as a framework to select an intermediate off-rate. A simplified mathematical model is employed to select starting concentrations of biotin-IL10 and avidin to investigate the effects of biomaterial properties. The release of biotinylated proteins from biomaterials depends on affinity interactions and diffusion (Equation 1):

$$\frac{\partial [ B ]}{\partial t} = \frac{D}{r} \frac{\partial }{\partial r} ( r \frac{\partial [ B ]}{\partial r} ) + D \frac{{\partial }^{2} [ B ]}{\partial {z}^{2}} - {k}_{on} [ A ] [ B ] + {k}_{off} ( [ AB ] )$$

where r is the radius and z is the height of a cylindrical biomaterial, [B] is the concentration of biotinylated IL10, measured using an IL10 ELISA, and [A] is the concentration of avidin or the avidin-like protein at any time t, measured using an avidin ELISA. The presence of biotinylated proteins does not impact the efficacy of ELISA. [AB] is the concentration of the avidin-biotin complex at time t, which can be determined by subtracting the concentration of released avidin [A] from the starting concentration [A]ₒ and assuming that any remaining avidin in the system is bound to biotin. The biotinylated IL10-avidin-biotinylated scaffold interaction is assumed to be a single bond to simplify the model. D is the diffusion coefficient of biotinylated IL10 in the scaffold, which can be determined experimentally by using non-biotinylated scaffolds and/or biotinylated scaffolds without avidin. The on and off rates (kₒₙ and k_{off}) are derived from SPR experiments in Example 10, or by approximation through fitting Equation 1 to release curves of biotinylated IL10, independently from the results of Example 10. The completed equation is used to describe the extent of control over protein release for a range of preparation parameters (FIG. 5).

Effects on Biomaterial Structure

The impact of biomaterial structure on release of biotinylated proteins is evaluated by comparing the release from biotinylated gelatin (a derivative of collagen) in three forms: coated on well plates (flat surface), in hydrogel (bulk 3D), or in the form of a porous scaffold (pseudo-3D). All materials are prepared from photocrosslinked methacrylated gelatin (GelMA) because of its versatility in controlling crosslinking parameters and previous work characterizing the impact of 3D printing on the mechanical properties of porous hydrogel scaffolds. For bulk hydrogel preparation, GelMA is biotinylated prior to hydrogel formation. For the 2D and pseudo-3D structures, GelMA is biotinylated after preparation so that only the surface is functionalized. The depth of functionalization into the materials is characterized for different durations of biotinylation reaction using fluorescent streptavidin binding (FIG. 1a). Given the same starting concentrations of avidin and biotinylated IL10, the release of IL10 is slowest from hydrogels, followed by porous scaffolds, followed by coatings. Equation 1 is implemented in three dimensions to understand the contribution of 3D structure and any accumulation on release kinetics.

Effects on Crosslinking

The effects of diffusion properties is studied by varying the crosslinking density of gelatin hydrogels by controlling the degree of substitution of methacrylate groups in the polymer backbone. Release is slower in more crosslinked hydrogels because of slower diffusion and thus greater availability of binding species over time. Thus, manipulation of affinity binding interactions in combination with 3D structure and diffusion properties allows control over protein release from biomaterials.

Release from Complex Biomaterials/Applications

To demonstrate the versatility of this platform technology, the release of IL10 from three complex biomaterials is controlled from which controlled release is currently not possible: engineered tissues, bioprosthetic heart valves, and titanium implants.

Biotinylation of engineered cartilage tissue is conducted. In the harsh environment of the arthritic joint, inflammatory cytokines and enzymes cause cartilage to undergo degradation, vascularization, and ossification. Thus, an immunomodulatory strategy to inhibit inflammation of tissue-engineered cartilage benefits the millions of people living with joint pain and disability worldwide.

Bioprosthetic heart valves prepared from glutaraldehyde-crosslinked bovine pericardium are biotinylated. These materials are currently used clinically, especially for pediatric patients, but suffer from inflammation-mediated calcification and failure as early as three years after implantation.

Titanium implants. Orthopedic implants that are currently in clinical use rapidly release BMP2 at such high doses that they may promote tumor growth. A controlled release strategy for these materials represents a major improvement.

Methods

Preparation of Model Biomaterials

Gelatin type A (Sigma Aldrich) is methacrylated to form GelMA as previously described. GelMA is dissolved in water and cast into well plates to form flat surfaces (˜100 um thick). Biopsy punches are used to obtain bulk 3D hydrogels from thicker coatings (˜2 mm in height). Porous hydrogel scaffolds will be prepared with pores of about 100μm in diameter with 99% interconnectivity and strut thickness of 100μm, using 3D bioprinting (Biobots, Philadelphia) according to our previously described methods using LAP as a photoinitiator activated by visible light.

For preparation of bioprosthetic heart valve material, pericardium from bovine tissue obtained from a local abattoir is crosslinked with 0.6% glutaraldehyde for 48 hours, followed by thorough rinsing. Engineered cartilage is prepared from bovine chondrocytes isolated from cartilage obtained from a local abattoir and cultured in agarose hydrogels for 6 weeks in chondrogenic cell culture media as previously described. Commercially available titanium substrates are aminated after silanization. All materials are then biotinylated using NHS chemistry as described in Example 10. IL10 is biotinylated through similar techniques as described in Example 10 through NHS chemistry to the terminal amine or to side chains containing lysine. These sites are not closely linked to the receptor binding site of IL10, and preliminary studies suggest that bioactivity of IL10 is retained after biotinylation (FIGS. 4A-4B), but bioactivity will be confirmed by adding to human macrophages and measuring gene expression (see below—Bioactivity Studies).

Release Studies

The release of biotinylated IL10 from the biomaterials is characterized over the course of several weeks or months as necessary in vitro using ELISA to measure the amount of released IL10 at regular time intervals. ELISA is unaffected by the presence of biotinylated proteins. Alternatively, IL10 can be fluorescently labeled with fluorescent isothiocyanate (FITC) for tracking via fluorescent spectroscopy. Release media is phosphate buffered saline (PBS) and cell culture media containing 10% human serum, which has been shown to more accurately recapitulate the in vivo environment. IL10 is covalently conjugated to the biomaterials through similar chemistries as a control in order to determine to what extent scaffold dissolution plays a role in the release profile. The biomaterials are degraded at the end of each release study with gelatinase to determine if any IL10 remains bound.

Bioactivity Studies

In all studies, bioactivity of released IL10 is evaluated by adding released samples adjusted to 40 ng/ml to human macrophages and measuring gene expression for markers of the M2c phenotype after 24-48 hrs. Primary human macrophages are prepared from peripheral blood-derived monocytes as in previous studies. M2c activation with IL10 from release studies is evaluated via quantitative RTPCR for gene expression, and proteins secreted into the media are measured by ELISA. Gene expression markers of M2c activation include CD163, MMP7, MMP8, SPP1, VCAN, and MARCO, and secreted proteins include MMP7 and MMP8.

Statistical Analysis

All experiments are conducted in triplicate. Nonlinear regression is used to determine the effective rate of release in each phase of protein release in order to compare the release kinetics of different formulations using ANOVA. A similar technique is used to analyze the effects of cellular composition on the kinetics of different phases of fibrin clot contraction.

Results and Alternative Strategies

Increasing the surface area available for binding as the protein diffuses from the biomaterial (through the use of porous scaffolds or hydrogels compared to flat surfaces) causes a decrease in the effective rate of release. Thus, by varying the starting concentrations and the 3D structure of the biomaterials, biomaterials are prepared with release profiles ranging from several days to several weeks. In this way, these experiments are completely independent from the results of Example 10, although development of the mathematical model will be complemented by determination of on- and off-rates in Example 10. With definition of these variables, the mathematical model in Equation 1 is predictive of the approximate release profiles, but may be modified with empirical measurements to reflect the complexity of the interactions between biotinylated IL10, avidin, and biotinylated gelatin.

These results provide evidence that any biomaterial or engineered tissue are transformed into a controlled release system using biotin-avidin interactions. The modularity of the design process, availability and versatility of reagents, and the development of a simple mathematical model ensure widespread adaptation of the technology to diverse applications.

### Example 12

### In Vivo Environmental Impacts on the Release of Biotinylated Proteins

The three main aspects of the in vivo microenvironment that impact biotin-avidin-mediated release are the presence of endogenous biotin (which would displace lower affinity biotin conjugates), the number of infiltrating macrophages (which degrade biomaterials), and the degree of vascularization (which would increase flow). These parameters are systematically evaluated using a combination of in vitro and in vivo experiments (Table 4).

TABLE 4
Experiments

| Experiment | Variables | Outcomes |
|---|---|---|
| Effects of | 0, 4, 400, and | Increasing rates of release in vitro |
| biotin | 4000 ng/ml | |
| concentration | biotin | |
| Effects of | 0, 2, 10 and 20 | Increasing rates of release in vitro |
| macrophages | million | |
| | macrophages | |
| Preliminary | Low dose IL10 | Faster release in vivo compared to in |
| in vivo | (0.1 ng/day), | vitro (in vivo imaging) |
| experiment | High dose IL10 | Promotion of the M2c phenotype |
| Correlation | (1 ng/day), | with IL10 release (immunostaining |
| with in vitro | No IL10 | and gene expression)-Increased blood |
| release | (negative | vessel density with IL10 release (in |
| profiles | control), and | vivo imaging and CD31 staining at 4 |
| Correlation | Covalently | weeks) |
| with | conjugated | Decreased fibrous capsule thickness |
| vascularity | IL10 (covalent | with IL10 release (Masson's |
| Effects on | control) | trichrome staining) |
| foreign body | | |
| response | | |

A preliminary in vivo experiment also tests the hypothesis that promoting M2c activation of infiltrating host macrophages enhances the integration and vascularization of implanted biomaterials. Fundamental insight into the role of the in vivo microenvironment on protein release is provided, and proof of concept that the host inflammatory response can be modulated through biomaterial design to enhance vascularization and integration is provided.

Effects of Endogenous Biotin In Vitro

Serum levels of endogenous biotin in mice have been reported to be 4 ng/ml. This concentration is about 20,000 times lower than the concentration of biotin conjugated to the scaffolds (FIG. 1). Nonetheless, the presence of free biotin, even at low concentrations, may have a significant impact on the release of biotin-IL10, and patient-to-patient variability in circulating biotin levels could affect the performance of biotin-avidin-mediated release systems. Therefore, the release of biotin-IL10 from porous hydrogel scaffolds is characterized in vitro in the presence of free biotin ranging from 0 to 4,000 ng/ml. Even low concentrations of biotin increase the rate of release of biotin-IL10 from the scaffolds in a dose-dependent fashion. This effect will be incorporated into the mathematical model described in Example 11 through addition of a term describing the rate of free biotin-mediated release, or kₒₙ[AB_{IL10}][B_{f}], from the reaction AB_{IL10}+B_{f}→AB_{f}+B_{IL10}, where A represents avidin, B_{f} represents free biotin, B_{IL10} represents biotinylated IL10, and the rate of reaction is the on-rate of free biotin and avidin (6.7×10⁷ M⁻¹s⁻¹)⁴⁵.

Effects of Infiltrating Macrophages In Vitro

Macrophages secrete high levels of MMPs, especially MMP9, which degrades gelatin and would therefore be expected to increase the rate of release of conjugated molecules. Release of fluorescently labeled IL10 is characterized over several weeks from scaffolds seeded with 0 to 20 million human macrophages, which covers the expected range of numbers of macrophages recruited upon implantation in mice. This effect is incorporated into the mathematical model through empirical determination of the effect of seeded macrophages on the crosslinking density of the materials and the diffusion coefficient of IL10.

Comparison of In Vitro and In Vivo Release Profiles

The release of fluorescently labeled biotin-IL10 is characterized in vivo using non-invasive imaging over the course of 4 weeks in a murine subcutaneous implantation model. This time point was chosen because minimal changes in the foreign body response to subcutaneously implanted polymeric biomaterials are typically observed after 4 weeks. Two different doses of IL10 (approximately 0.1 and 1 ng per day) are tested in comparison to a negative control containing no IL10 and another control containing covalently conjugated IL10, to confirm that release is required for functionality in vivo. Release profiles of IL10 is determined from in vivo imaging and compared to in vitro release profiles of scaffolds described above.

Effects of Vascularization on Release Profiles

At each time point for in vivo imaging, the mice are infused via tail vein injection with a solution of fluorescent dextran for determination of the degree of vascularity in the vicinity of the implanted biomaterials. The release rate of IL10 increases with vascularity because of increased flow, causing deviations from in vitro release profiles.

Effects of Released IL10 on Inflammatory Response After 4 weeks of non-invasively tracking the in vivo release of IL10, scaffolds are explanted for analysis of the effects of IL10 release on the foreign body response and macrophage behavior. Half of each scaffold is used for histological analysis of fibrous capsule thickness and immunohistochemical analysis of blood vessels (CD31) and markers of macrophage phenotype. The other half of the scaffolds is used for gene expression analysis, allowing a more quantitative analysis of macrophage phenotype via a panel of 120 genes related to the M1, M2a, and M2c phenotypes.

Scaffold Preparation

Biotinylated IL10 are tagged with FITC by attaching it via maleimide crosslinking to thiol groups in one of its four cysteine-containing side chains, or via NHS chemistry to the terminal amine or to side chains containing lysine, and bioactivity is confirmed according to the methods described in Example 11. Biotin-IL10 is incorporated into 3D porous gelatin hydrogel scaffolds using the methods described in Example 11.

Negative control scaffolds are also prepared with biotinylation and avidin but without biotinylated IL10. Another negative control group with covalently conjugated IL10 is prepared using standard EDC/NHS chemistry.

Subcutaneous Implantation Model and In Vivo Imaging

Surgeries and veterinary care are conducted according to the Guide for the Care and Use of Laboratory Animals and protocols approved by the Institutional Animal Care and Use Committee (IACUC) at Drexel University, including procedures for limiting discomfort, distress, and pain, and the AVMA Guidelines on Euthanasia. One scaffold from each group is implanted subcutaneously in C57B¹/₆ mice (n=6) because they are commonly used to study the foreign body response to biomaterials and because the similarities and differences between macrophages from these mice and those from humans have been extensively studied. Also, up to 4 scaffolds can be implanted into one mouse without conflicting effects on macrophage behavior. Six replicates provides enough statistical power for in vivo imaging and for gene expression and histological analysis. At predetermined intervals after implantation (days 0, 3, 6, 9,12, 14, 21, and 28), mice are scanned using the IVIS in vivo imaging system for fluorescence in the GFP channel (488/509 nm). The fluorescence intensity of each sample (4 scaffolds per mouse) is calculated from the fluorescence signal, corrected with the GFP background filters to minimize tissue autofluorescence, and plotted over time to characterize the kinetics of release.

Analysis of the Inflammatory Response

After 4 weeks of implantation, half of each scaffold (together with any surrounding fibrous capsule) is fixed and prepared for immunohistochemical analysis, and the other half is used for gene expression analysis. Samples are sectioned and stained with triple immunofluorescence for qualitative analysis of markers of the M1, M2a, and M2c phenotypes, including the pan-macrophage marker F4/80 in combination with two other markers of macrophage phenotype, including iNOS and CCR7 for M1; Arg1 and CD206 for M2a; and CD163 and VCAN for M2c. For quantitative analysis of macrophage phenotype via gene expression, RNA is extracted and gene expression analysis is performed using a custom-designed Nanostring code set for 120 genes that constitute the signatures of murine M1, M2a, and M2c macrophages (40 genes per phenotype). Because macrophages are the dominant cell type making up the fibrous capsule, there is no need to sort cells prior to RNA extraction for gene expression analysis.

Statistical analysis

In vitro and in vivo release kinetics are assessed in triplicate and compared using nonlinear regression to describe the effective rates of each phase of release followed by ANOVA, as described in Example 11. For the effects on fibrous capsule thickness, blood vessel density, and macrophage phenotype markers (n=6), the effects of IL10 release profile on each parameter are compared using a one way ANOVA corrected for multiple comparisons.

Results and Alternative Strategies

The release profiles of IL10 in vivo are faster than those measured in vitro, even when endogenous biotin and seeded macrophages are taken into account, because animal movement and flow cannot be accounted for in vitro. It is important to note that even if 4 weeks of release cannot be achieved using biotin-avidin interactions, the evaluation of the effects of the microenvironment on even short-term release profiles will still be highly valuable. The sustained release of IL10 in vivo will be associated with enhanced M2c activation of infiltrating macrophages, diminished fibrous encapsulation and increased numbers of blood vessels. Future work will focus on proving causation of biomaterial-mediated activation of the M2c phenotype on vascularization, and on optimization of dose and timing.

This study provides 1) fundamental insight into the effects of the in vivo microenvironment on affinity-based release, 2) proof of concept that the biotin-avidin strategy can be used to control the release of proteins in vivo, and 3) proof of concept that the in vivo manipulation of host macrophage phenotype can be used to enhance biomaterial success. These results pave the way for strategies to harness, rather than inhibit, the inflammatory response in regenerative medicine.

### References:

1. N. M. Green, “Thermodynamics of the binding of biotin and some analogues by avidin,” Biochem J, vol. 101, pp. 774-80, December 1966.

2. N. M. Green, “Avidin,” Adv Protein Chem, vol. 29, pp. 85-133, 1975.

3. M. H. Qureshi and S. L. Wong, “Design, production, and characterization of a monomeric streptavidin and its application for affinity purification of biotinylated proteins,” Protein Expr Purif, vol. 25, pp. 409-15, August 2002.

4. S. Metzger, P. S. Lienemann, C. Ghayor, W. Weber, I. Martin, F. E. Weber, et al., “Modular poly(ethylene glycol) matrices for the controlled 3D-localized osteogenic differentiation of mesenchymal stem cells,” Adv Healthc Mater, vol. 4, pp. 550-8, Mar. 11, 2015.

5. B. Taskinen, D. Zauner, S. I. Lehtonen, M. Koskinen, C. Thomson, N. Kahkonen, et al., “Switchavidin: reversible biotin-avidin-biotin bridges with high affinity and specificity,” Bioconjug Chem, vol. 25, pp. 2233-43, Dec. 17, 2014.

6. K. Kaiser, M. Marek, T. Haselgrubler, H. Schindler, and H. J. Gruber, “Basic studies on heterobifunctional biotin-PEG conjugates with a 3-(4-pyridyldithio)propionyl marker on the second terminus,” Bioconjug Chem, vol. 8, pp. 545-51, July-August 1997.

7. C. E. Chivers, E. Crozat, C. Chu, V. T. Moy, D. J. Sherratt, and M. Howarth, “A streptavidin variant with slower biotin dissociation and increased mechanostability,” Nat Methods, vol. 7, pp. 391-3, May 2010.

8. E. Bruneau, D. Sutter, R. I. Hume, and M. Akaaboune, “Identification of nicotinic acetylcholine receptor recycling and its role in maintaining receptor density at the neuromuscular junction in vivo,” J Neurosci, vol. 25, pp. 9949-59, Oct. 26, 2005.

9. O. H. Laitinen, H. R. Nordlund, V. P. Hytonen, and M. S. Kulomaa, “Brave new (strept)avidins in biotechnology,” Trends Biotechnol, vol. 25, pp. 269-77, June 2007.

10. C. Xie, L. Ma, J. Liu, X. Li, H. Pei, M. Xiang, et al., “SKLB023 blocks joint inflammation and cartilage destruction in arthritis models via suppression of nuclear factor-kappa B activation in macrophage,” PLoS One, vol. 8, p. e56349, 2013.

11. G. Huybrechts-Godin, P. Hauser, and G. Vaes, “Macrophage-fibroblast interactions in collagenase production and cartilage degradation,” Biochem J, vol. 184, pp. 643-50, Dec. 15, 1979.

12. E. G. Lima, A. R. Tan, T. Tai, L. Bian, A. M. Stoker, G. A. Ateshian, et al., “Differences in interleukin-1 response between engineered and native cartilage,” Tissue Eng Part A, vol. 14, pp. 1721-30, October 2008.

13. J. M. Low-Marchelli, V. C. Ardi, E. A. Vizcarra, N. van Rooijen, J. P. Quigley, and J. Yang, “Twist1 induces CCL2 and recruits macrophages to promote angiogenesis,” Cancer Res, vol. 73, pp. 662-71, Jan. 15, 2013.

14. A. Fantin, J. M. Vieira, G. Gestri, L. Denti, Q. Schwarz, S. Prykhozhij, et al., “Tissue macrophages act as cellular chaperones for vascular anastomosis downstream of VEGF-mediated endothelial tip cell induction,” Blood, vol. 116, pp. 829-40, Aug. 5, 2010.

15. Y. Kubota, K. Takubo, T. Shimizu, H. Ohno, K. Kishi, M. Shibuya, et al., “M-CSF inhibition selectively targets pathological angiogenesis and lymphangiogenesis,” J Exp Med, vol. 206, pp. 1089-102, May 11, 2009.

16. L. M. Arendt, J. McCready, P. J. Keller, D. D. Baker, S. P. Naber, V. Seewaldt, et al., “Obesity Promotes Breast Cancer by CCL2-Mediated Macrophage Recruitment and Angiogenesis,” Cancer Res, vol. 73, pp. 6080-6093, Oct. 1, 2013.

17. E. Sakurai, A. Anand, B. K. Ambati, N. van Rooijen, and J. Ambati, “Macrophage depletion inhibits experimental choroidal neovascularization,” Invest Ophthalmol Vis Sci, vol. 44, pp. 3578-85, August 2003.

18. N. Hibino, T. Yi, D. R. Duncan, A. Rathore, E. Dean, Y. Naito, et al., “A critical role for macrophages in neovessel formation and the development of stenosis in tissue-engineered vascular grafts,” FASEB J, vol. 25, pp. 4253-63, December 2011.

19. K. L. Spiller, S. L, C. E. Witherel, R. R. Anfang, J. Ng, K. R. Nakazawa, et al., “Sequential delivery of immunomodulatory cytokines to facilitate the M1-to-M2 transition of macrophages and enhance vascularization of bone scaffolds,” Biomaterials, vol. 37, pp. 194-207, January 2015.

20. K. L. Spiller, R. R. Anfang, K. J. Spiller, J. Ng, K. R. Nakazawa, J. W. Daulton, et al., “The role of macrophage phenotype in vascularization of tissue engineering scaffolds,” Biomaterials, vol. 35, pp. 4477-88, May 2014.

21. “National Science Board's Science and Engineering Indicators 2014,” 2014.

22. M. P. Xiong, M. L. Forrest, A. L. Karls, and G. S. Kwon, “Biotin-triggered release of poly(ethylene glycol)-avidin from biotinylated polyethylenimine enhances in vitro gene expression,” Bioconjug Chem, vol. 18, pp. 746-53, May-June 2007.

23. N. Oliva, M. Carcole, M. Beckerman, S. Seliktar, A. Hayward, J. Stanley, et al., “Regulation of dendrimer/dextran material performance by altered tissue microenvironment in inflammation and neoplasia,” Sci Transl Med, vol. 7, p. 272ra11, Jan. 28, 2015.

24. Z. Lu, G. Wang, C. R. Dunstan, Y. Chen, W. Y. Lu, B. Davies, et al., “Activation and promotion of adipose stem cells by tumour necrosis factor-alpha preconditioning for bone regeneration,” J Cell Physiol, vol. 228, pp. 1737-44, August 2013.

25. E. A. Vermeij, M. G. Broeren, M. B. Bennink, O. J. Arntz, I. Gjertsson, L. E. M. v. L. P, et al., “Disease-regulated local IL-10 gene therapy diminishes synovitis and cartilage proteoglycan depletion in experimental arthritis,” Ann Rheum Dis, Jul. 15, 2014.

26. T. Hemmerle, F. Doll, and D. Neri, “Antibody-based delivery of IL4 to the neovasculature cures mice with arthritis,” Proc Natl Acad Sci U S A, vol. 111, pp. 12008-12, Aug. 19, 2014.

27. K. L. Spiller, Y. Liu, J. L. Holloway, S. A. Maher, Y. Cao, W. Liu, et al., “A novel method for the direct fabrication of growth factor-loaded microspheres within porous nondegradable hydrogels: Controlled release for cartilage tissue engineering,” J Control Release, vol. 157, pp. 39-45, Jan. 10, 2012.

28. M. J. Thubrikar, J. D. Deck, J. Aouad, and S. P. Nolan, “Role of mechanical stress in calcification of aortic bioprosthetic valves,” J Thorac Cardiovasc Surg, vol. 86, pp. 115-25, July 1983.

29. E. R. Mohler, 3rd, F. Gannon, C. Reynolds, R. Zimmerman, M. G. Keane, and F. S. Kaplan, “Bone formation and inflammation in cardiac valves,” Circulation, vol. 103, pp. 1522-8, Mar. 20, 2001.

30. D. A. Towler, “Molecular and cellular aspects of calcific aortic valve disease,” Circ Res, vol. 113, pp. 198-208, Jul. 5, 2013.

31. B. D. Ratner, “Reducing capsular thickness and enhancing angiogenesis around implant drug release systems,” J Control Release, vol. 78, pp. 211-8, Jan. 17, 2002.

32. T. Hisatome, Y. Yasunaga, S. Yanada, Y. Tabata, Y. Ikada, and M. Ochi, “Neovascularization and bone regeneration by implantation of autologous bone marrow mononuclear cells,” Biomaterials, vol. 26, pp. 4550-6, August 2005.

33. N. Hirose, H. Maeda, M. Yamamoto, Y. Hayashi, G. H. Lee, L. Chen, et al., “The local injection of peritoneal macrophages induces neovascularization in rat ischemic hind limb muscles,” Cell Transplant, vol. 17, pp. 211-22, 2008.

34. J. D. Roh, R. Sawh-Martinez, M. P. Brennan, S. M. Jay, L. Devine, D. A. Rao, et al., “Tissue-engineered vascular grafts transform into mature blood vessels via an inflammation-mediated process of vascular remodeling,” Proc Natl Acad Sci U S A, vol. 107, pp. 4669-74, Mar. 9, 2010.

35. A. Sindrilaru, T. Peters, S. Wieschalka, C. Baican, A. Baican, H. Peter, et al., “An unrestrained proinflammatory M1 macrophage population induced by iron impairs wound healing in humans and mice,” J Clin Invest, vol. 121, pp. 985-97, March 2011.

36. Y. Wang, Y. P. Wang, G. Zheng, V. W. Lee, L. Ouyang, D. H. Chang, et al., “Ex vivo programmed macrophages ameliorate experimental chronic inflammatory renal disease,” Kidney Int, vol. 72, pp. 290-9, August 2007.

37. K. L. Spiller, S. Nassiri, C. E. Witherel, R. Anfang, J. Ng, K. Nakazawa, et al., “Sequential delivery of cytokines to facilitate the M1-to-M2 transition of macrophages and enhance vascularization of bone scaffolds,” Biomaterials, vol. 37, pp. 194-207, 2015.

38. C. M. Champagne, J. Takebe, S. Offenbacher, and L. F. Cooper, “Macrophage cell lines produce osteoinductive signals that include bone morphogenetic protein-2,” Bone, vol. 30, pp. 26-31, January 2002.

39. M. B. Schmidt, E. H. Chen, and S. E. Lynch, “A review of the effects of insulin-like growth factor and platelet derived growth factor on in vivo cartilage healing and repair,” Osteoarthritis Cartilage, vol. 14, pp. 403-12, May 2006.

40. L. C. Tetlow, D. J. Adlam, and D. E. Woolley, “Matrix metalloproteinase and proinflammatory cytokine production by chondrocytes of human osteoarthritic cartilage: associations with degenerative changes,” Arthritis Rheum, vol. 44, pp. 585-94, March 2001.

41. D. T. Felson, R. C. Lawrence, P. A. Dieppe, R. Hirsch, C. G. Helmick, J. M. Jordan, et al., “Osteoarthritis: new insights. Part 1: the disease and its risk factors,” Ann Intern Med, vol. 133, pp. 635-46, Oct. 17, 2000.

42. “National and state medical expenditures and lost earnings attributable to arthritis and other rheumatic conditions—United States, 2003,” MMWR Morb Mortal Wkly Rep, vol. 56, pp. 4-7, Jan. 12, 2007.

43. D. T. Felson and Y. Zhang, “An update on the epidemiology of knee and hip osteoarthritis with a view to prevention,” Arthritis Rheum, vol. 41, pp. 1343-55, August 1998.

44. C. C. f D. C. a. Prevention). (2010). Arthritis. Meeting the Challenge: At A Glance 2010. Available: http://www.cdc.gov/chronicdisease/resources/publications/AAG/arthritis.htm

45. S. D. Ramsey, K. Newton, D. Blough, D. K. McCulloch, N. Sandhu, G. E. Reiber, et al., “Incidence, outcomes, and cost of foot ulcers in patients with diabetes,” Diabetes Care, vol. 22, pp. 382-7, March 1999.

46. S. Nassiri, I. Zakeri, M. S. Weingarten, and K. L. Spiller, “Relative expression of pro-inflammatory and anti-inflammatory genes reveals differences between healing and nonhealing human chronic diabetic foot ulcers.,” Journal of Investigative Dermatology, 2015;135(6):1700-3.

47. J. Khallou-Laschet, A. Varthaman, G. Fornasa, C. Compain, A. T. Gaston, M. Clement, et al., “Macrophage plasticity in experimental atherosclerosis,” PLoS One, vol. 5, p. e8852, 2010.

48. K. A. Kigerl, J. C. Gensel, D. P. Ankeny, J. K. Alexander, D. J. Donnelly, and P. G. Popovich, “Identification of two distinct macrophage subsets with divergent effects causing either neurotoxicity or regeneration in the injured mouse spinal cord,” J Neurosci, vol. 29, pp. 13435-44, Oct. 28, 2009.

49. S. Sahebjam, R. Khokha, and J. S. Mort, “Increased collagen and aggrecan degradation with age in the joints of Timp3(−/−) mice,” Arthritis Rheum, vol. 56, pp. 905-9, March 2007.

50. J. G. Bolivar, S. A. Soper, and R. L. McCarley, “Nitroavidin as a ligand for the surface capture and release of biotinylated proteins,” Anal Chem, vol. 80, pp. 9336-42, Dec. 1, 2008.

51. K. Vulic, M. M. Pakulska, R. Sonthalia, A. Ramachandran, and M. S. Shoichet, “Mathematical model accurately predicts protein release from an affinity-based delivery system,” J Control Release, vol. 197, pp. 69-77, Jan. 10, 2015.

52. J. D. Hirsch, L. Eslamizar, B. J. Filanoski, N. Malekzadeh, R. P. Haugland, J. M. Beechem, et al., “Easily reversible desthiobiotin binding to streptavidin, avidin, and other biotin-binding proteins: uses for protein labeling, detection, and isolation,” Anal Biochem, vol. 308, pp. 343-57, Sep. 15, 2002.

53. H. Gopi, S. Cocklin, V. Pirrone, K. McFadden, F. Tuzer, I. Zentner, et al., “Introducing metallocene into a triazole peptide conjugate reduces its off-rate and enhances its affinity and antiviral potency for HIV-1 gp120,” J Mol Recognit, vol. 22, pp. 169-74, Mar-April 2009.

54. A. Emileh, C. Duffy, A. P. Holmes, A. Rosemary Bastian, R. Aneja, F. Tuzer, et al., “Covalent conjugation of a peptide triazole to HIV-1 gp120 enables intramolecular binding site occupancy,” Biochemistry, vol. 53, pp. 3403-14, Jun. 3, 2014.

55. H. Vashisth and C. F. Abrams, “All-atom structural models of insulin binding to the insulin receptor in the presence of a tandem hormone-binding element,” Proteins, vol. 81, pp. 1017-30, June 2013.

56. C. F. Abrams and E. Vanden-Eijnden, “Large-scale conformational sampling of proteins using temperature-accelerated molecular dynamics,” Proc Natl Acad Sci U S A, vol. 107, pp. 4961-6, Mar. 16, 2010.

57. B. Kuhn and P. A. Kollman, “Binding of a diverse set of ligands to avidin and streptavidin: an accurate quantitative prediction of their relative affinities by a combination of molecular mechanics and continuum solvent models,” J Med Chem, vol. 43, pp. 3786-91, Oct. 5, 2000.

58. B. Kuhn and P. A. Kollman, “A Ligand That Is Predicted to Bind Better to Avidin than Biotin: Insights from Computational Fluorine Scanning,” J Am Chem Soc, vol. 122, pp. 3909-3916, 2000.

59. N. M. Green and M. A. Joynson, “A preliminary crystallographic investigation of avidin,” Biochem J, vol. 118, pp. 71-2, June 1970.

60. K. Hofmann, G. Titus, J. A. Montibeller, and F. M. Finn, “Avidin binding of carboxyl-substituted biotin and analogues,” Biochemistry, vol. 21, pp. 978-84, Mar. 2, 1982.

61. A. Emileh, F. Tuzer, H. Yeh, M. Umashankara, D. R. Moreira, J. M. Lalonde, et al., “A model of peptide triazole entry inhibitor binding to HIV-1 gp120 and the mechanism of bridging sheet disruption,” Biochemistry, vol. 52, pp. 2245-61, Apr. 2, 2013.

62. S. C. Meyer, T. Gaj, and I. Ghosh, “Highly selective cyclic peptide ligands for NeutrAvidin and avidin identified by phage display,” Chem Biol Drug Des, vol. 68, pp. 3-10, July 2006.

63. M. D. Wood, G. H. Borschel, and S. E. Sakiyama-Elbert, “Controlled release of glial-derived neurotrophic factor from fibrin matrices containing an affinity-based delivery system,” J Biomed Mater Res A, vol. 89, pp. 909-18, Jun. 15, 2009.

64. M. Rusckowski, M. Fogarasi, B. Fritz, and D. J. Hnatowich, “Effect of endogenous biotin on the applications of streptavidin and biotin in mice,” Nucl Med Biol, vol. 24, pp. 263-8, April 1997.

65. I. Gadjanski, S. Yodmuang, K. Spiller, S. Bhumiratana, and G. Vunjak-Novakovic, “Supplementation of exogenous adenosine 5′-triphosphate enhances mechanical properties of 3D cell-agarose constructs for cartilage tissue engineering,” Tissue Eng Part A, vol. 19, pp. 2188-200, October 2013.

66. A. J. Schuerwegh, E. J. Dombrecht, W. J. Stevens, J. F. Van Offel, C. H. Bridts, and L. S. De Clerck, “Influence of pro-inflammatory (IL-1 alpha, IL-6, TNF-alpha, IFN-gamma) and anti-inflammatory (IL-4) cytokines on chondrocyte function,” Osteoarthritis Cartilage, vol. 11, pp. 681-7, September 2003.

67. W. B. Tsai and M. C. Wang, “Effects of an avidin-biotin binding system on chondrocyte adhesion and growth on biodegradable polymers,” Macromol Biosci, vol. 5, pp. 214-21, Mar. 15, 2005.

68. J. F. Woessner, Jr., “The determination of hydroxyproline in tissue and protein samples containing small proportions of this imino acid,” Arch Biochem Biophys, vol. 93, pp. 440-7, May 1961.

69. I. Gadjanski, K. Spiller, and G. Vunjak-Novakovic, “Time-dependent processes in stem cell-based tissue engineering of articular cartilage,” Stem Cell Rev, vol. 8, pp. 863-81, September 2012.

70. K. L. Spiller, J. L. Holloway, M. E. Gribb, and A. M. Lowman, “Design of semi-degradable hydrogels based on poly(vinyl alcohol) and poly(lactic-co-glycolic acid) for cartilage tissue engineering,” J Tissue Eng Regen Med, vol. 5, pp. 636-47, August 2011.

71. D. Mendis, I. Ginon, H. Louis, J. L. McGregor, and R. N. Poston, “Cell biotinylation provides a sensitive and effective detection technique for cellular adhesion assays: comparison with existing methods,” J Immunol Methods, vol. 253, pp. 57-68, Jul. 1, 2001.

72. G. J. Bancroft, R. D. Schreiber, and E. R. Unanue, “Natural immunity: a T-cell-independent pathway of macrophage activation, defined in the scid mouse,” Immunol Rev, vol. 124, pp. 5-24, December 1991.

73. I. f L. A. R. Committee for the Update of the Guide for the Care and Use of Laboratory Animals, National Research Council of the National Academies, Guide for the Care and Use of Laboratory Animals: Eight Edition. Washington, D.C.: The National Academies Press, 2010.

74. P. M. Steed, M. G. Tansey, J. Zalevsky, E. A. Zhukovsky, J. R. Desjarlais, D. E. Szymkowski, et al., “Inactivation of TNF signaling by rationally designed dominant-negative TNF variants,” Science, vol. 301, pp. 1895-8, Sep. 26, 2003.

75. “National Science Board's Science and Engineering Indicators 2012,” 2012.

76. K. McGlynn. (2012). The Plot Thickens: Teaching Statistics Using Math Games. Available: http://tip.drupalgardens.com/sites/g/files/g764316/f/201308/McGlynn, Klair-Unit_0_0.pdf

77. (2013). Society for Biomaterials Education Challenge. Available: http://2013.biomaterials.org/node/100

78. Curriculum standards, Philadelphia School District, Courtesy of Ms. Klair McGlynn

79. International Patent Application No. WO 2015/077401.

80. D. K. Cullen, M. D. Tang-Schomer, L. A. Struzyna, A. R. Patel, V. E. Johnson, J. A. Wolf, et al., “Microtissue engineered constructs with living axons for targeted nervous system reconstruction,” Tissue Eng Part A, vol. 18, pp. 2280-9, November 2012.

81. Remington's Pharmaceutical Sciences (1985, Genaro, ed., Mack Publishing Co., Easton, Pa.

82. Rolny C, Mazzone M, Tugues S, Laoui D, Johansson I, Coulon C, Squadrito M L, Segura I, Li X, Knevels E, Costa S, Vinckier S, Dresselaer T, Akerud P, De Mol M, Salomaki H, Phillipson M, Wyns S, Larsson E, Buysschaert I, Botling J, Himmelreich U, Van Ginderachter J A, De Palma M, Dewerchin M, Claesson-Welsh L, Carmeliet P. HRG inhibits tumor growth and metastasis by inducing macrophage polarization and vessel normalization through downregulation of P1GF. Cancer Cell. 2011;19(1):31-44.

83. Quiding-Jarbrink M, Raghavan S, Sundquist M. Enhanced M1 macrophage polarization in human helicobacter pylori-associated atrophic gastritis and in vaccinated mice. PLoS One. 2010;5(11):e15018.

84. Mirza R, Koh T J. Dysregulation of monocyte/macrophage phenotype in wounds of diabetic mice. Cytokine. 2011;56(2):256-64.

85. Brown B N, Valentin J E, Stewart-Akers A M, McCabe G P, Badylak S F. Macrophage phenotype and remodeling outcomes in response to biologic scaffolds with and without a cellular component. Biomaterials. 2009;30(8):1482-91.

86. Furukawa S, Moriyama M, Tanaka A, Maehara T, Tsuboi H, Iizuka M, Hayashida J N, Ohta M, Saeki T, Notohara K, Sumida T, Nakamura S. Preferential M2 macrophages contribute to fibrosis in IgG4-related dacryoadenitis and sialoadenitis, so-called Mikulicz's disease. Clin Immunol. 2015;156(1):9-18.

87. Fuentes-Duculan J, Suarez-Farinas M, Zaba L C, Nograles K E, Pierson K C, Mitsui H, Pensabene C A, Kzhyshkowska J, Krueger J G, Lowes M A. A subpopulation of CD163-positive macrophages is classically activated in psoriasis. J Invest Dermatol. 2010;130(10):2412-22.

88. National Science Board's Science and Engineering Indicators 2014. 2014.

89. Fryer R G, Levitt S D. Understanding the black-white test score gap in the first two years of school. Review of Economics and Statistics. 2004;86.2:447-64.

90. Reynolds A J, Temple J A, Ou S R, Arteaga I A, White B A. School-based early childhood education and age-28 well-being: effects by timing, dosage, and subgroups. Science. 2011;333(6040):360-4.

91. Green NM. Thermodynamics of the binding of biotin and some analogues by avidin. Biochem J. 1966;101(3):774-80.

92. Clark R, Olson K, Fuh G, Marian M, Mortensen D, Teshima G, Chang S, Chu H, Mukku V, Canova-Davis E, Somers T, Cronin M, Winkler M, Wells JA. Long-acting growth hormones produced by conjugation with polyethylene glycol. J Biol Chem. 1996;271(36):21969-77.

93. Jabbarzadeh E, Starnes T, Khan Y M, Jiang T, Wirtel A J, Deng M, Lv Q, Nair L S, Doty S B, Laurencin C T. Induction of angiogenesis in tissue-engineered scaffolds designed for bone repair: a combined gene therapy-cell transplantation approach. Proc Natl Acad Sci U S A. 2008;105(32):11099-104.

94. Gueret P, Combe S, Krezel C, Fuseau E, van Giersbergen P L, Petitou M, Neuhart E. First in man study of EP217609, a new long-acting, neutralisable parenteral antithrombotic with a dual mechanism of action. Eur J Clin Pharmacol. 2016.

95. Sur S, Fries A C, Kinzler K W, Zhou S, Vogelstein B. Remote loading of preencapsulated drugs into stealth liposomes. Proc Natl Acad Sci U S A. 2014;111(6):2283-8.

96. Arendt L M, McCready J, Keller P J, Baker D D, Naber S P, Seewaldt V, Kuperwasser C. Obesity Promotes Breast Cancer by CCL2-Mediated Macrophage Recruitment and Angiogenesis. Cancer Res. 2013;73(19):6080-93.

97. Sakurai E, Anand A, Ambati B K, van Rooijen N, Ambati J. Macrophage depletion inhibits experimental choroidal neovascularization. Invest Ophthalmol Vis Sci. 2003;44(8):3578-85.

98. Madden L R, Mortisen D J, Sussman E M, Dupras S K, Fugate J A, Cuy J L, Hauch K D, Laflamme M A, Murry C E, Ratner B D. Proangiogenic scaffolds as functional templates for cardiac tissue engineering. Proc Natl Acad Sci U S A. 2010;107(34):15211-6.

99. Hsu S M, Raine L. Protein A, avidin, and biotin in immunohistochemistry. J Histochem Cytochem. 1981;29(11):1349-53.

100. Bigini P, Previdi S, Casarin E, Silvestri D, Violatto M B, Facchin S, Sitia L, Rosato A, Zuccolotto G, Realdon N, Fiordaliso F, Salmona M, Morpurgo M. In vivo fate of avidin-nucleic acid nanoassemblies as multifunctional diagnostic tools. ACS Nano. 2014;8(1):175-87.

101. Edwards A, Civitello A, Hammond H A, Caskey C T. DNA typing and genetic mapping with trimeric and tetrameric tandem repeats. Am J Hum Genet. 1991;49(4):746-56.

102. Hofmann K, Finn F M. Receptor affinity chromatography based on the avidin-biotin interaction. Ann N Y Acad Sci. 1985;447:359-72.

103. DeJarnette N. America's Children: Providing Early Exposure to STEM (Science, Technology, Engineering and Math) Initiatives. Education. 2012;133:77-84.

104. Starkey P, Klein A, Wakeley A. Enhancing young children's mathematical knowledge through a pre-kindergarten mathematics intervention. Early Childhood REsearch Quarterly. 2004;19(1):99-120.

105. Bagiati A, Yoon S Y, Evangelou D, Ngambeki I. Engineering Curricula in Early Education: Describing the Landscape of Open Resources. Early Childhood Research & Practice. 2010;12(2):1-15.

106. Malicki R, Potts D. The outcomes of outbound student mobility. AIM Overseas. 2013:1-9.

107. Di Pietro G. Do Study Abroad Programs Enhance the Employability of Graduates? Education Finance and Policy. 2015;10:223-43.

108. Mitchell BS, Vogler M, Nerad M, editors. EVALUATING INTERNATIONAL RESEARCH EXPERIENCES FOR GRADUATE STUDENTS. CGS-NSF-DFG WORKSHOP; 2016; National Science Foundation.

109. Yu T, Wang W, Nassiri S, Kwan T, Dang C, Liu W, Spiller KL. Temporal and spatial distribution of macrophage phenotype markers in the foreign body response to glutaraldehyde-crosslinked gelatin hydrogels. J Biomater Sci Polym Ed. 2016;27(8):721-42.

110. Lurier E B, Dalton D, Dampier W, Nassiri S, Raman P, Rajagopalu R, Sarmady M, Spiller K L. Role of IL10-stimulated macrophages in the early stages of wound healing. In preparation for submission to the Journal of Immunology. 2016.

111. Lu J Y, Cao Q, Zheng D, Sun Y, Wang C, Yu X, Wang Y, Lee V W, Zheng G, Tan T K, Wang X, Alexander S I, Harris D C, Wang Y. Discrete functions of M and M macrophage subsets determine their relative efficacy in treating chronic kidney disease. Kidney Int. 2013.

112. Ardi V C, Kupriyanova T A, Deryugina E I, Quigley J P. Human neutrophils uniquely release TIMP-free MMP-9 to provide a potent catalytic stimulator of angiogenesis. Proc Natl Acad Sci U S A. 2007;104(51):20262-7.

113. Gabriel V A, McClellan E A, Scheuermann RH. Response of human skin to esthetic scarification. Burns. 2014;40(7):1338-44.

114. Bronneke S, Bruckner B, Sohle J, Siegner R, Smuda C, Stab F, Wenck H, Kolbe L, Gronniger E, Winnefeld M. Genome-wide expression analysis of wounded skin reveals novel genes involved in angiogenesis. Angiogenesis. 2015;18(3):361-71.

115. Greco J A, 3rd, Pollins A C, Boone B E, Levy S E, Nanney L B. A microarray analysis of temporal gene expression profiles in thermally injured human skin. Burns. 2010;36(2):192-204.

116. Arnold L, Henry A, Poron F, Baba-Amer Y, van Rooijen N, Plonquet A, Gherardi R K, Chazaud B. Inflammatory monocytes recruited after skeletal muscle injury switch into antiinflammatory macrophages to support myogenesis. J Exp Med. 2007;204(5):1057-69.

117. Schlundt C, El Khassawna T, Serra A, Dienelt A, Wendler S, Schell H, van Rooijen N, Radbruch A, Lucius R, Hartmann S, Duda G N, Schmidt-Bleek K. Macrophages in bone fracture healing: Their essential role in endochondral ossification. Bone. 2015.

118. Zizzo G, Hilliard B A, Monestier M, Cohen P L. Efficient clearance of early apoptotic cells by human macrophages requires M2c polarization and MerTK induction. J Immunol. 2012;189(7):3508-20.

119. Fickenscher H, Hor S, Kupers H, Knappe A, Wittmann S, Sticht H. The interleukin-10 family of cytokines. Trends Immunol. 2002;23(2):89-96.

120. Roohani-Esfahani S I, Dunstan C R, Davies B, Pearce S, Williams R, Zreiqat H. Repairing a critical-sized bone defect with highly porous modified and unmodified baghdadite scaffolds. Acta Biomater. 2012;8(11):4162-72.

121. Li J J, Roohani-Esfahani S I, Dunstan C R, Quach T, Steck R, Saifzadeh S, Pivonka P, Zreiqat H. Efficacy of novel synthetic bone substitutes in the reconstruction of large segmental bone defects in sheep tibiae. Biomed Mater. 2016;11(1):015016.

122. Roohani-Esfahani S I, Dunstan C R, Li J J, Lu Z, Davies B, Pearce S, Field J, Williams R, Zreiqat H. Unique microstructural design of ceramic scaffolds for bone regeneration under load. Acta Biomater. 2013;9(6):7014-24.

123. Graney P L, Roohani-Esfahani I, Zreiqat H, Spiller K L. Response of macrophages to biomaterials used in bone regeneration. Journal of the Royal Society Interface. 2016;in press.

124. Graney P L, Roohani-Esfahani I, Zreiqat H, Spiller K L. In vitro modulation of macrophage behavior by ceramic-based scaffolds. Annual Meeting of the Society For Biomaterials. 2015.

125. Graney P L, Roohani-Esfahani I, Nassiri S, Zreiqat H, Spiller K L. Biomaterial-mediated control over macrophage behavior in bone regeneration. Tissue Engineering Part A 2015;21(S1):S106.

126. Graney P L, Roohani-Esfahani I, Nassiri S, Zreiqat H, Spiller K L. Biomaterial-mediated modulation of macrophage behavior to promote bone regeneration. World Biomaterials Congress. 2016.

127. Shandalov Y, Egozi D, Koffler J, Dado-Rosenfeld D, Ben-Shimol D, Freiman A, Shor E, Kabala A, Levenberg S. An engineered muscle flap for reconstruction of large soft tissue defects. Proc Natl Acad Sci USA. 2014;111(16):6010-5.

128. Bolivar J G, Soper S A, McCarley R L. Nitroavidin as a ligand for the surface capture and release of biotinylated proteins. Anal Chem. 2008;80(23):9336-42.

129. Laitinen O H, Nordlund H R, Hytonen V P, Kulomaa M S. Brave new (strept)avidins in biotechnology. Trends Biotechnol. 2007;25(6):269-77.

130. Chen Y C, Lin R Z, Qi H, Yang Y, Bae H, Melero-Martin J M, Khademhosseini A. Functional Human Vascular Network Generated in Photocrosslinkable Gelatin Methacrylate Hydrogels. Adv Funct Mater. 2012;22(10):2027-39.

131. Ersumo N, Spiller K L. Differences in time-dependent mechanical properties between extruded and molded hydrogels. In revisions at Biofabrication. 2016.

132. Van Den Bulcke A I, Bogdanov B, De Rooze N, Schacht E H, Cornelissen M, Berghmans H. Structural and rheological properties of methacrylamide modified gelatin hydrogels. Biomacromolecules. 2000;1(1):31-8.

133. Ghazanfari S, Driessen-Mol A, Hoerstrup S P, Baaijens F P, Bouten C V. Collagen Matrix Remodeling in Stented Pulmonary Arteries after Transapical Heart Valve Replacement. Cells Tissues Organs. 2016;201(3):159-69.

134. Oomen P J, Loerakker S, van Geemen D, Neggers J, Goumans M J, van den Bogaerdt A J, Bogers A J, Bouten C V, Baaijens F P. Age-dependent changes of stress and strain in the human heart valve and their relation with collagen remodeling. Acta Biomater. 2016;29:161-9.

135. MacGrogan D, Luxan G, Driessen-Mol A, Bouten C, Baaijens F, de la Pompa J L. How to make a heart valve: from embryonic development to bioengineering of living valve substitutes. Cold Spring Harb Perspect Med. 2014;4(11):a013912.

136. Manji R A, Zhu L F, Nijjar N K, Rayner D C, Korbutt G S, Churchill T A, Rajotte R V, Koshal A, Ross D B. Glutaraldehyde-fixed bioprosthetic heart valve conduits calcify and fail from xenograft rejection. Circulation. 2006;114(4):318-27.

137. Carragee E J, Hurwitz E L, Weiner B K. A critical review of recombinant human bone morphogenetic protein-2 trials in spinal surgery: emerging safety concerns and lessons learned. Spine J. 2011;11(6):471-91.

138. Akkan C K, Hur D, Uzun L, Garipcan B. Amino acid conjugated self assembling molecules for enhancing surface wettability of fiber laser treated titanium surfaces. Applied Surface Science. 2016;366:284-91.

139. Bhat R, Sell S, Wagner R, Zhang J T, Pan C, Garipcan B, Boland W, Bossert J, Klemm E, Jandt K D. The Janus-SAM approach for the flexible functionalization of gold and titanium oxide surfaces. Small. 2010;6(3):465-70.

140. Garipcan B, Winters J, Atchison J S, Cathell M D, Schiffman J D, Leaffer O D, Nonnenmann S S, Schauer C L, Piskin E, Nabet B, Spanier J E. Controllable formation of nanoscale patterns on TiO2 by conductive-AFM nanolithography. Langmuir. 2008;24(16):8944-9.

141. Gorbahn M, Klein M O, Lehnert M, Ziebart T, Brullmann D, Koper I, Wagner W, Al-Nawas B, Veith M. Promotion of osteogenic cell response using quasicovalent immobilized fibronectin on titanium surfaces: introduction of a novel biomimetic layer system. J Oral Maxillofac Surg. 2012;70(8):1827-34.

142. Wohl-Bruhn S, Badar M, Bertz A, Tiersch B, Koetz J, Menzel H, Mueller P P, Bunjes H. Comparison of in vitro and in vivo protein release from hydrogel systems. J Control Release. 2012;162(1):127-33.

143. Witherel C E, Graney P L, Freytes D O, Weingarten M S, Spiller K L. Response of human macrophages to wound matrices in vitro. Wound Repair and Regeneration. 2016;in press.

144. Tutwiler V, Litvinov R I, Lozhkin A P, Peshkova A D, Lebedeva T, Ataullakhanov F I, Spiller K L, Cines D B, Weisel J W. Kinetics and mechanics of clot contraction are governed by the molecular and cellular composition of the blood. Blood. 2016;127(1):149-59.

145. Mooney J E, Rolfe B E, Osborne G W, Sester D P, van Rooijen N, Campbell G R, Hume D A, Campbell J H. Cellular plasticity of inflammatory myeloid cells in the peritoneal foreign body response. Am J Pathol. 2010;176(1):369-80.

146. Peppas N A, Merrill E W. Crosslinked poly(vinyl alcohol) hydrogels as swollen elastic networks. Journal of Applied Polymer Science. 1977;21:1763-70.

146. Zhang L, Cao Z, Bai T, Carr L, Ella-Menye J R, Irvin C, Ratner B D, Jiang S. Zwitterionic hydrogels implanted in mice resist the foreign-body reaction. Nat Biotechnol. 2013;31(6):553-6.

148. Zhu Y, Lyapichev K, Lee D, Motti D, Ferraro N, Yahn S, Soderblom C, Zha J, Bethea J R, Spiller K L, Lemmon V, Lee J. Macrophage-specific translational profile reveals lipid catabolic pathways after spinal cord injury. In review at EMBO Molecular Medicine. 2016.

149. Committee for the Update of the Guide for the Care and Use of Laboratory Animals IfLAR, National Research Council of the National Academies. Guide for the Care and Use of Laboratory Animals: Eight Edition. Washington, D.C.: The National Academies Press; 2010.

150. Spiller K L, Wrona E A, Romero-Torres S, Pallotta I, Graney P L, Witherel C E, Panicker L M, Feldman R A, Urbanska A M, Santambrogio L, Vunjak-Novakovic G, Freytes D O. Differential Gene Expression in Human, Murine, and Cell Line-derived Macrophages upon Polarization. Exp Cell Res. 2015.

151. Schroder K, Irvine K M, Taylor M S, Bokil N J, Le Cao K A, Masterman K A, Labzin L I, Semple C A, Kapetanovic R, Fairbairn L, Akalin A, Faulkner G J, Baillie J K, Gongora M, Daub C O, Kawaji H, McLachlan G J, Goldman N, Grimmond S M, Carninci P, Suzuki H, Hayashizaki Y, Lenhard B, Hume D A, Sweet M J. Conservation and divergence in Toll-like receptor 4-regulated gene expression in primary human versus mouse macrophages. Proc Natl Acad Sci U S A. 2012;109(16):E944-53.

152. Martinez F O, Helming L, Milde R, Varin A, Melgert B N, Draijer C, Thomas B, Fabbri M, Crawshaw A, Ho L P, Ten Hacken N H, Cobos Jimenez V, Kootstra N A, Hamann J, Greaves D R, Locati M, Mantovani A, Gordon S. Genetic programs expressed in resting and IL-4 alternatively activated mouse and human macrophages: similarities and differences. Blood. 2013;121(9):e57-69.

153. Cheryan S, Siy J O, Vichayapai M, Drury B J, Kim S. Do Female and Male Role Models Who Embody STEM Stereotypes Hinder Women's Anticipated Success in STEM? Social Psychological and Personality Science. 2011;2(6):656-64.

154. The School District of Philadelphia Office of Curriculum IaA. Science and Social Studies and World Language (K-8). 2013 [cited 2016].

Any document (including but not limited to any patent, patent application, publication, and website) listed herein is hereby incorporated herein by reference in its entirety. While these developments have been disclosed with reference to specific embodiments, it is apparent that other embodiments and variations of this invention are devised by others skilled in the art without departing from the true spirit and scope of the developments. The appended claims include such embodiments and variations thereof.

1. A method of modulating the immune response to a tissue scaffold, comprising biotinylating IL4 or IL10, biotinylating a scaffold, and linking the biotinylated IL4 or IL10 to the scaffold via avidin or streptavidin.

2. The method according to claim 1, wherein said IL4 or IL10 is biotinylated via a spacer arm.

3. The method according to claim 1, wherein said spacer arm is sulfo-NHS, Sulfo-NHS-LC, Sulf-NHS-LC-LC, NHS-PEG4, or NHS-PEG12.

4. The method according to claim 1, wherein said biotinylation is with biotin.

5. The method according to claim 1, wherein said biotinylation is with desthiobiotin or iminobiotin.

6. The method according to claim 1, wherein the tissue scaffold is for cartilage.

7. The method according to claim 1, wherein the tissue scaffold is for bone.

8. A method of delivering IL4 or IL10 to a macrophage, comprising biotinylating IL4 or IL10, biotinylating a nanoparticle, linking the biotinylated IL4 or IL10 to the nanoparticle, and delivering the nanoparticle to the macrophage.

9. The method according to claim 8, wherein said IL4 or IL10 is biotinylated via a spacer arm.

10. The method according to claim 8, wherein said spacer arm is sulfo-NHS, Sulfo-NHS-LC, Sulf-NHS-LC-LC, NHS-PEG4, or NHS-PEG12.

11. The method according to claim 8, wherein said biotinylation is with biotin.

12. The method according to claim 8, wherein said biotinylation is with desthiobiotin or iminobiotin.

13. A method of modulating the immune response to a tissue scaffold, comprising biotinylating interferon gamma, biotinylating a scaffold, and linking the biotinylated interferon gamma via avidin or streptavidin.

14. The method according to claim 13, further comprising biotinylating IL4 or IL10 and linking the biotinylated IL4 or IL10 to the scaffold via avidin or streptavidin.
